(19) United States
(12) Patent Application Publication
(10) Pub. No.: US 2026/0284379 A1
(43) Pub. Date: Sep. 24, 2026

(54) POLYMER COMPOSITE DRY ELECTRODES FOR ELECTRICAL STIMULATION AND METHODS OF USE THEREOF

(71) Applicant: UNIVERSITY HEALTH NETWORK, Toronto (CA)

(72) Inventors: Milos R POPOVIC, Mississauga (CA); Hani E NAGUIB, North York (CA); Zia SAADATNIA, North York (CA); Melissa MARQUEZ CHIN, Toronto (CA); Baptiste GARNIER, Toronto (CA)

(21) Appl. No.: 19/168,243

(22) PCT Filed: Mar. 25, 2024
(86) PCT No.: PCT/CA2024/050368

§ 371 Date: Sep. 23, 2025

Related U.S. Application Data
(60) Provisional application No. 63/454,498, filed on Mar. 24, 2023.
(60) Provisional application No. 63/619,703, filed on Jan. 10, 2024.

(51) Int. Cl. A61N 1/04; A61N 1/36; C08J 3/205; C08J 5/12; C08K 3/04; D04B 1/16; D04B 1/24; H01B 1/24

(52) U.S. Cl. CPC A61N 1/0496; A61N 1/0452; A61N 1/0476; A61N 1/0484; A61N 1/36003; A61N 1/36034; C08J 3/2053; C08J 5/128; C08K 3/041; D04B 1/16; D04B 1/24; H01B 1/24; C08J 2327/16; C08K 2201/001; C08K 2201/011; C08K 2201/016; D10B 2331/04

(57) ABSTRACT

Electrodes known in the art and used with functional electrical stimulation degrade after multiple uses and require constant wetting or a skin interface layer to be comfortable. A reusable, convenient and comfortable dry electrode as herein disclosed comprises a conductive material comprising a fluoropolymer matrix and conductive carbon nanoparticles dispersed in the matrix, and a conductor configured to contact and deliver an electrical pulse to the material from a stimulator, wherein a dry tissue-contacting surface the material is configured to deliver electrical stimulation directly to a tissue. The present disclosure also relates to uses of reusable dry electrodes, dry electrode garments and devices and methods of manufacturing and uses thereof.

### RELATED APPLICATIONS

This Patent Cooperation Treaty application claims the benefit of priority of U.S. Provisional Application 63/454,498 filed on Mar. 24, 2023, and U.S. Application No. 63/619,703 filed on Jan. 10, 2024, each of which are incorporated herein in their entirety.

### FIELD

The present disclosure relates to reusable dry electrodes. The present disclosure also relates to methods of manufacturing washable dry electrode articles or garments. The present disclosure also relates to dry electrode devices. The present disclosure also relates to uses of reusable dry electrodes and dry electrode articles, garments and devices.

### BACKGROUND

Functional electrical stimulation (FES) is a rehabilitation technique that enables functional improvements in patients with motor control impairments and consists of delivering low energy electrical pulses through peripheral nerves of an affected limb to activate targeted muscles [5] to produce muscle contractions which can be used for rehabilitation of people with upper motor neuron injury such as stroke and spinal cord injury which often result in paralysis [1]-[3], [6]-[7]]. These pulses are delivered through electrodes which become the main interface between the stimulator and a patient. In order to deliver stimulation, the minimum setup requirements are a stimulator and at least a pair of electrodes. Electrodes can be placed on the top of the skin (i.e., transcutaneous), through the skin (i.e., percutaneous), directly on the nerve (i.e., cuff), or directly on the muscle next to the nerve (i.e., epimysial) [1], [2]). Their charge transfer can be influenced by many factors, including placement, the tissue-electrode interface, the electrode material, and the stimulator used, which can ultimately affect movement production and comfort sensations during stimulation [4],[8,9]. These factors can also play a role in the user's experience, being it the patient or the therapist.

The stimulator is the device which provides the electrical pulses. The pulses can be of different shapes, widths, and amplitudes, and can be delivered at specific frequencies. The electrodes are the interface between the stimulator and the body. They can be categorized into invasive or non-invasive depending on whether they are placed directly on the nerve, through the skin, or on the surface of the skin. Invasive electrodes require a surgical procedure for implantation and deliver stimulation directly to a specific nerve or close to a motor neuron. Non-invasive electrodes are placed on the skin (i.e., transcutaneous) and the electrical impulses are delivered through the skin's surface [1], [2]. Within the transcutaneous electrode category, the current standard for FES are self-adhesive hydrogel electrodes. These are multi-layered electrodes with an adhesive hydrogel in contact with the skin [5]. They are easy to use but tend to lose their adhesive properties in the short-term which makes them not ideal for reuse.

Transcutaneous electrodes have the benefits of being non-invasive and easy to apply and remove. The types of transcutaneous electrodes used to date include sponge electrodes, carbon rubber electrodes, hydrogel electrodes, and textile electrodes [5].

One of the most commonly used type of transcutaneous electrode is the self-adhesive hydrogel electrode. Their wide use is understandable given their non-invasive nature and easy application. They consist of multiple layers of material which include an adhesive gel that allows it to adhere directly to the skin [5]. These types of electrodes, however, have the disadvantage of degrading after multiple uses making it lose its adhesive properties quickly. In order to keep the electrodes in place, an elastic bandage or other additional supports can be wrapped around the electrode. This prevents the electrodes from falling off due to body movement, tugging on the cable connecting the electrode to the stimulator, or the weight of the cable connecting the electrode to the stimulator. Hydrogel electrodes are also not reusable, which makes them more expensive to use if multiple sessions are needed.

The way by which stimulation is typically delivered is cumbersome, as it requires at least two hydrogel electrodes per stimulated muscle, which are linked to the stimulator using long cables [10,11]. This is not a major issue for the stimulation of one or two muscles, but to achieve more complex tasks, such as reaching, grasping and walking, that involve 6 to 12 stimulation electrode pairs, the use of hydrogel electrodes and individual cables is prohibitively cumbersome. Consequently, more sophisticated forms of FES therapy must be delivered in a clinical setting, as they require a knowledgeable therapist to deliver the treatment. Thus, a new method for delivering FES therapies is needed.

To address this problem, various groups started developing dry electrodes, with the objective of using them as reusable stimulation electrodes [12]. One of the benefits of dry electrodes is that they are reusable and can be used without the need for conductive gel. A wide range of materials can be used to create dry electrodes [13], making them easily adaptable for FES applications [22, 23, 29]. However, one of the fundamental problems with dry electrodes, as metal plate or carbon rubber, is that they are uncomfortable and individuals using them feel pain and discomfort during stimulation [17]. Indeed, due to their high conductivity, metal plate electrodes can cause severe skin burns and require to be associated with a wet medium to homogeneously distribute the current. Similarly, carbon rubber electrodes, another type of non-adhesive transcutaneous electrode made from carbon and elastomers such as silicone which have low conductivity, require gel or water as skin interface and repeated wetting, and the use of additional straps, tape, or wraps to hold them in place [5], making them difficult to use for long term applications.

Recently, textile electrodes have been proposed as a solution for some of the hydrogel electrode limitations, however for use with electrical stimulation or functional electrical stimulation, they require constant wetting or a skin interface layer in order to be comfortable, which makes them less convenient to use compared to hydrogel electrodes [5], [16]-[21]. For example, embroidered electrode pads made of plasma-coated metallized yarn [5] have been used with electrical stimulation, however they could not be comfortably used with higher currents (8 mA or above) without a skin interface layer due to improper electrical properties (e.g. impedance) and ineffective skin-electrode interfaces. A hotmelt-carbon composite made from thermoplastic polymer (e.g., polyolefins or polyamides) has been used as an interface between textile electrodes and skin to alleviate intensity of stimulation from high current delivery [5]. In such situations, the hotmelt-carbon composite functions as an interfacial layer between the stimulation electrodes and the skin to improve impedance matching, and does not function as an electrode itself which would require an interfacial layer between the electrode and skin to ensure efficient and comfortable stimulation [5].

The level of comfort may affect users' tolerance and adherence to stimulation, so a higher comfort level could potentially enable the use of FES for longer periods of time or to produce stronger muscle contractions. Since current transcutaneous electrode options require the use of a wet interface to improve performance and increase comfort, the constant addition of gel or water can become impractical and uncomfortable for users.

Garments (e.g., shirts, pants) knitted with a conductive Ag—AgCl and LYCRA-coated nylon yarn have been used with functional electrical stimulation, however they required consistent moistening of the fabric electrodes to be comfortably and reliably used [8].

Conductive polymer composites are robust structures fabricated by the combination of thermoplastic polymers and conductive nano fillers providing unique properties with high mechanical flexibility and durability [22]. Conductive polymer films containing conductive carbon nanoparticles dispersed in fluoropolymer matrix have been used as force sensors designed to be wrapped around neurosurgical tools. For example, a 200 μm-thick tube was wrapped in conductive polymer film and covered with interdigitated electrode and lamination layers for interfacing with and receiving pressure signals from silicon-based simulated neural tissue [23]. Conductive carbon nanotube composites made up of carbon nanotubes (CNTs) dispersed in thermoplastic polyurethane (TPU), ethylene-vinyl acetate (EVA), and styrene-butadiene-styrene (SBS) have been used for acquiring electrical signals from heart tissue during electrocardiograms (ECGs) [22].

To make the FES more convenient, different wearable devices have been proposed [20, 24]. Progress in printing technology has enabled the development of printed electrodes on a textile substrate [25]. However, the porous nature of textile materials makes it difficult to control the amount of ink and, thus the electrode properties [26]. On the other hand, knitting technology enables the development of garments, such as sleeves or tights, integrating textile conductive yarns to create textile electrodes in specific locations [18]. However, developed textile electrodes are not suitable for FES applications due their discomfort. Despite better performances in terms of flexibility or stretchability, they also require, at least, to be wet before stimulation [20]. Moreover, conventional textile materials and processes produce rough electrode surfaces that don't match the skin's roughness and impedance [27]. The same applies to embroidery technology, except that the electrode is sewn onto the substrate after its fabrication [28].

Dry electrodes for use with electrical stimulation that are convenient, comfortable and/or reusable, and wearable devices for FES that addresses any of the above discussed challenges, namely, (i) reusability, (ii) minimized pain and discomfort, (iii) easy donning and doffing, and/or (iv) simplified connection to a stimulator for electric pulse transfer, are desirable.

### SUMMARY

The present disclosure relates to reusable dry electrodes and uses thereof, methods of manufacturing washable dry electrode articles or garments, and uses of dry electrode articles, garments and devices.

The present disclosure also relates to use of a dry polymer composite electrode, for example based on polyvinylidene fluoride (PVDF) thermoplastics and carbon nanotube (CNT) conductive fillers for FES on a knitted substrate to provide a smart garment. The electrode and stimulator are integrated and connected through a textile conductive matrix, made of sewn or embroidered textile conductive yarns. Thus, the described methods of manufacture of reusable dry electrodes can be applied to produce smart FES garments for a wide range of muscles, which are wearable and suitable for long term applications such as FES therapy.

In one embodiment, a reusable dry electrode comprises a conductive material comprising a fluoropolymer matrix and conductive carbon nanoparticles dispersed in the matrix, and a conductor configured to contact and deliver an electrical pulse to the material from a stimulator, wherein a dry tissue-contacting surface of the material is configured to deliver electrical stimulation directly to a tissue, for example, without passing through an intervening layer disposed between the tissue-contacting surface and the tissue. The conductive material is, for example, non-metallic (e.g., the conductive material does not use metal for electrical conduction).

In another embodiment, the fluoropolymer matrix is polyvinylidene fluoride (PVDF).

In another embodiment, the nanoparticles are carbon nanotubes (CNTs).

In another embodiment, the nanoparticles are dispersed in the fluoropolymer matrix at about 5 to about 10 wt % of the matrix.

In another embodiment, the nanoparticles have an aspect ratio of about 1 to about 600.

In another embodiment, the aspect ratio of the nanoparticles is about 130 to about 160.

In another embodiment, the nanoparticles are interconnected.

In another embodiment, impedance of the conductive material is about 1,000 to about 10,000Ω and/or sheet resistivity of the conductive material is at least 50 Ω·cm or about 50 to about 500 Ω·cm.

In another embodiment, impedance of the conductive material is about 2,100 to about 2,800Ω and/or the sheet resistivity of the conductive material is about 100 to about 300 Ω·cm.

In another embodiment, the conductive material has a width of about 50 to about 200 μm.

In another embodiment, the width of the conductive material is about 50 to about 100 μm.

In another embodiment, the tissue contacting the dry electrode is skin.

In another embodiment, the dry electrode is adapted for positioning on skin in proximity to a selected muscle or group thereof.

In another embodiment, the electrical stimulation induces a contraction of a muscle or group thereof.

In another embodiment, the electrical stimulation is functional electrical stimulation.

In another embodiment, a dry electrode is disposed on an article or a garment.

In another embodiment, a dry electrode is disposed on an inside surface of an article or garment such to juxtapose a dry tissue-contacting surface of the electrode against a tissue.

In another embodiment, a dry electrode is affixed to an article or garment with an adhesive.

In another embodiment, a dry electrode is used with a high-rise voltage-regulated stimulator, wherein the electrode is configured to deliver an electrical pulse.

In another embodiment, a pulse used with a dry electrode is a biphasic electrical pulse that is symmetric or asymmetric.

In another embodiment, rise time of the pulse is about 10 to about 40 ns.

In another embodiment, the pulse has a total width of about 8 to about 2,000 μs.

In another embodiment, the width of the pulse is about 100 to about 1,000 μs.

In another embodiment, the width is about 300 to about 500 μs.

In another embodiment, the pulse is asymmetric and comprises a first phase pulse having a first amplitude and a first width and a second phase pulse having a second amplitude and a second width.

In another embodiment, the first amplitude of the pulse is about −0.04 to about −160 mA and/or the second amplitude of the pulse is about 0.01 to about 40 mA.

In another embodiment, the first amplitude of the pulse is about −0.4 to about −120 mA and/or the second amplitude of the pulse is about 0.1 to about 30 mA.

In another embodiment, the first width of the pulse is about 200 to about 500 μs and/or the second width of the pulse is about 800 to about 2,000 μs.

In another embodiment, the first and second amplitudes of the pulse are unequal.

In another embodiment, the first and second widths of the pulse are unequal.

In another embodiment, a washable dry electrode article or garment comprises a dry electrode as herein disclosed.

In another embodiment, a dry electrode is disposed on an inside surface of an article or garment such to juxtapose a tissue-contacting surface of the conductive material against a tissue.

In another embodiment, a dry electrode is affixed to an article or garment with an adhesive.

In another embodiment, the conductor comprises an electrode-contacting segment configured for contacting the dry electrode, the electrode-contacting segment comprising at least one conductive yarn integrated into a knitted textile substrate.

In another embodiment, the at least one conductive yarn is integrated into the textile substrate via sewing or embroidery.

In another embodiment, the conductor comprises at least two conductive yarns and portions of the conductive yarns are integrated into the textile substrate to form a pattern comprising a plurality of non-intersecting lines.

In another embodiment, the conductor further comprises a cable segment comprising non-integrated portions of the conductive yarn or yarns configured for delivering electrical stimulation to the conductor electrode-contacting segment from a stimulator, optionally wherein the cable segment is secured to the textile substrate by an attachment means.

In another embodiment, lengths of at least two of the plurality of non-intersecting lines are arranged substantially parallel relative to each other within the electrode-contacting segment.

In another embodiment, the textile substrate is or comprises polyester jersey fabric.

In another embodiment, the conductive yarn or yarns is/are or comprise silver-plated yarns and/or have a linear resistance of at least 1 Ω/m or about 1 to about 1000 Ω/m.

In another embodiment, a protective layer is disposed on a non-skin-contacting surface of the article or garment to cover exposed portions of the conductive yarn or yarns.

In another embodiment, the protective layer is a fabric layer or a liquid silicone layer.

In another embodiment, a dry electrode device for delivering electrical stimulation to a user comprises a dry electrode article or garment as herein disclosed, a high-rise voltage-regulated stimulator, and a connector linking the conductor and the stimulator.

In another embodiment, the dry electrode is affixed to an article or garment with an adhesive.

In another embodiment, a connector of a dry electrode is disposed on an article or garment.

In another embodiment, a stimulator used with a dry electrode, article, garment or device is portable.

In another embodiment, the stimulator is configured to be positioned within a portion of the article or garment.

In another embodiment, a dry electrode or dry electrode article, garment or device as herein disclosed is used for treating a mobility impairment in a user.

In another embodiment, uses of a dry electrode or dry electrode article, garment or device as herein disclosed comprises positioning the article, garment, or at least two dry electrodes directly on the tissue of the user in proximity to a selected muscle or group thereof, connecting the conductor of the article, garment or the at least two dry electrodes to the stimulator, and delivering electrical stimulation from the stimulator to the conductor of the article or garment or to the at least two dry electrodes, wherein the stimulation induces a contraction of the muscle or group thereof to treat a mobility impairment.

In another embodiment, the mobility impairment is paralysis.

In another embodiment, a method of delivering electrical stimulation to a subject as herein disclosed comprises positioning a dry electrode article or garment or at least two dry electrodes as herein defined directly on the tissue of a subject in proximity to a selected muscle or group thereof, connecting the conductors of the article, garment or the at least two dry electrodes to a high-rise voltage-regulated stimulator, and delivering an electrical pulse from the stimulator to the conductors of the article or garment or to the at least two dry electrodes, wherein the stimulation induces a contraction of the muscle or group thereof.

In another embodiment, the pulse is a biphasic electrical pulse that is symmetric or asymmetric.

In another embodiment, rise time of the pulse is about 10 to about 40 ns.

In another embodiment, the pulse has a total width of about 8 to about 2,000 μs.

In another embodiment, the width of the pulse is about 100 to about 1,000 μs.

In another embodiment, the width of the pulse is about 300 to about 500 μs.

In another embodiment, the pulse is asymmetric and comprises a first phase pulse having a first amplitude and a first width and a second phase pulse having a second amplitude and a second width.

In another embodiment, the first amplitude of the pulse is about −0.04 to about −160 mA and/or the second amplitude of the pulse is about 0.01 to about 40 mA.

In another embodiment, the first amplitude of the pulse is about −0.4 to about −120 mA and/or the second amplitude of the pulse is about 0.1 to about 30 mA.

In another embodiment, the first width of the pulse is about 200 to about 500 μs and/or the second width of the pulse is about 800 to about 2,000 μs.

In another embodiment, the first and second amplitudes of the pulse are unequal.

In another embodiment, the first and second widths of the pulse are unequal.

In another embodiment, the pulse is delivered at a frequency of about 1 to about 100 Hz.

In another embodiment, the frequency of the pulse is about 20 to about 40 Hz.

In another embodiment, a method of delivering electrical stimulation as herein disclosed is used for treating a mobility impairment in a subject.

In another embodiment, a method of manufacturing a washable dry electrode article or garment comprises A) fabricating a dry electrode as herein disclosed, the steps comprising i) dispersing conductive carbon nanoparticles into a polymer solvent to yield a dispersion, ii) dissolving fluoropolymer matrix in the dispersion to yield a blend, iii) casting the blend, iv) drying the casted blend, v) compressing the blend, vi) cooling the blend to yield a conductive material, vii) cleaning the material, and viii) disposing a conductor on or in the conductive material, wherein the conductor is configured to contact and deliver an electrical pulse to the material from a stimulator, and further comprises B) disposing the dry electrode of (A) on or in an article or a garment, wherein the electrode is disposed on an inside surface of the article or garment such to juxtapose the tissue-contacting surface of the electrode against the tissue, for example, without passing through an intervening layer disposed between the tissue-contacting surface and the tissue.

In another embodiment, the casting and/or compressing comprises transferring the blend into a mold.

In another embodiment, the solvent is dimethylformamide (DMF).

In another embodiment, the dispersing is effected by sonication.

In another embodiment, the dissolving, casting and/or drying further comprises heating the blend.

In another embodiment, the heating is effected within about 80 to about 200° C.

In another embodiment, the cleaning is effected with water and/or ethanol.

In another embodiment, the disposing a conductor on the material according to (viii) comprises integrating at least one conductive yarn into a knitted textile substrate.

In another embodiment, the integrating comprises sewing or embroidering.

In another embodiment, the disposing the dry electrode according to (B) further comprises i) positioning a thermoadhesive on the textile substrate before the integrating of the at least one conductive yarn, ii) positioning the dry electrode on the thermoadhesive, and iii) affixing the thermoadhesive to the dry electrode and textile substrate using thermal compression, wherein the thermal compression integrates a portion of the at least one conductive yarn into the dry electrode conductive material.

In another embodiment, the thermal compression comprises heating the dry electrode to a temperature of at least 180° C., optionally to a temperature between the range of 180° C. to 230° C.

In another embodiment, the thermal compression comprises applying a pressure between the range of 1 to 10 tons to the textile substrate, optionally applying about 1 ton of pressure followed by applying about 5 tons of pressure to the textile substrate.

In another embodiment, the pressure is applied for a time within the range of 5 to 10 minutes.

In another embodiment, the about 1 ton of pressure is applied for about 5 minutes and the about 5 tons of pressure is applied for about 5 to about 7 minutes.

In another embodiment, the textile substrate is or comprises polyester jersey fabric.

In another embodiment, the at least one conductive yarn is or comprises silver-plated yarn and/or has a resistance of at least 1 Ω·m or about 1 to about 1000 Ω·m.

In another embodiment, a protective layer is disposed on a non-skin-contacting surface of the article or garment to cover exposed portions of the at least one conductive yarn.

In another embodiment, the protective layer is a fabric layer or a liquid silicone layer.

In another embodiment, a dry electrode article or garment as manufactured by a method as herein disclosed is used for treating a mobility impairment in a user.

In another embodiment, the mobility impairment is paralysis.

Other features and advantages of the present disclosure will become apparent from the following detailed description. It should be understood, however, that the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

Embodiments of the present disclosure will now be described in relation to the drawings, in which:

FIG. 1 shows reusable dry electrodes as herein disclosed.

FIG. 2 shows the manufacturing process of a reusable dry electrode as herein disclosed.

FIGS. 3a and 3b shows TGA curves under two environments: FIG. 3a shows air and FIG. 3b Nitrogen. FIG. 3c shows tensile stress-strain curves of the dry electrode polymer nanocomposite and pristine polymer.

FIG. 4 shows impedance magnitude (top) and phase response (bottom) of the hydrogel, dry polymer nanocomposite, and dry carbon rubber electrodes on a person's forearm.

FIG. 5 shows examples of asymmetric biphasic pulses delivered using three different stimulators: a portable battery-operated stimulator (left: EV-906, Everyway Medical Instruments), a MyndSearch stimulator (middle: MyndTec, Canada), and a Compex Motion stimulator (right: Compex, Switzerland).

FIG. 6 shows the experimental setup, visual analog comfort rating scale, different electrode types used from left to right: hydrogel, dry polymer nanocomposite, and carbon rubber and stimulators used with their respective pulse shape from left to right: EV-906, MyndSearch, and Compex Motion.

FIG. 7 shows average stimulation intensities used for each electrode and stimulator type used. In each boxplot, the horizontal line indicates the median and the circle indicates the mean.

FIG. 8 shows average percentage of stimulation intensities used based on each stimulator's maximum possible output. In each boxplot, the horizontal line indicates the median and the circle indicates the mean.

FIG. 9 shows average comfort ratings reported, where 0 is the most comfortable and 10 the most uncomfortable, for each intensity level.

FIG. 10 shows each individual participant's comfort ratings for each combination of electrode-stimulator. 0=most comfortable and 10=most uncomfortable. The darker colors indicate higher comfort.

FIG. 11 shows average comfort ratings vs. intensity of stimulation used per electrode-stimulator combination.

FIG. 12 shows average normalized muscle torque generated during different stimulation intensities.

FIG. 13 shows each individual participant's normalized torques for each combination of electrode-stimulator. The darker colors indicate higher torque.

FIG. 14 shows average normalized generated torque vs. intensity of stimulation used per electrode-stimulator combination.

FIG. 15 shows average levels of sensations reported to be felt during stimulation.

FIG. 16 shows each individual participant's reported sensations for each combination of electrode-stimulator. Darker colors indicate the sensation was more highly perceived.

FIG. 17 shows combined scores of sensations per cutaneous (superficial), muscular (deep), and general categorization for each electrode type and stimulator used.

FIG. 18 shows average amount of normalized torque generated in relation to its perceived comfort level.

FIG. 19 shows 3D-plots showing average amount of torque generated vs. comfort rating vs. intensity of stimulation used at each intensity level.

FIG. 20 shows conductive material attached to the inside surface of a dry electrode garment as herein disclosed.

FIG. 21 shows the experimental setup used during electrical stimulation testing of a dry electrode garment as herein disclosed.

FIGS. 22 A and B show mean comfort ratings (FIG. 22B) obtained using the experimental setup and comfort rating scale shown in FIG. 22A for low (left), moderate (middle), and high (right) stimulation intensities with three different stimulators (EV-906, MyndSearch, and Compex Motion). A rating of 0 is very comfortable and 10 is very uncomfortable.

FIG. 23 shows a textile stitching pattern of a dry electrode article or garment sleeve.

FIGS. 24a and b show a sewn conductor matrix on a textile substrate and a nonwoven thermoadhesive. FIG. 24a shows an inner side of the conductor matrix (facing skin) and FIG. 24b shows a non-skin facing outer side of the conductor matrix.

FIGS. 25a-c show a process for attaching a dry electrode to a textile substrate. FIG. 25a shows conductive yarns are sewn onto a knitted and nonwoven thermoadhesive, then a dry electrode is fixed to the thermoadhesive using thermal compression. FIG. 25b shows an outer non-skin facing side of the integrated yarns before affixation of a dry electrode thereto, and FIG. 25c shows an inner skin-facing side of the integrated yarns after affixation of a dry electrode thereto.

FIG. 26a-c show a dry electrode article or garment sleeve for biceps. FIG. 26a shows an inner skin-facing side of the sleeve, FIG. 26b shows an outer non-skin facing side of the sleeve, and FIG. 26c shows the sleeve worn by a user and connected to a stimulator.

FIG. 27 shows average perceived comfort rating, wherein 0=most comfortable and 10=most uncomfortable.

FIG. 28 shows average normalized torque induced with stimulation using hydrogel electrodes vs. dry electrodes incorporated into wearable dry electrode sleeve garments.

FIG. 29 shows average stimulation intensity values used across all participants using hydrogel electrodes vs. dry electrodes incorporated into wearable dry electrode sleeve garments.

FIG. 30 shows average reported sensations after stimulation with hydrogel electrodes vs. dry electrodes incorporated into wearable dry electrode sleeve garments.

FIG. 31a shows a fabric pattern of a forearm dry electrode garment sleeve for functional electrical stimulation (FES), FIG. 31b shows a forearm dry electrode garment sleeve showing the dry electrode positions, FIG. 31c shows a zipper enabling closing of the sleeve, and FIG. 31d shows the sleeve worn by a user on a forearm.

FIG. 32 a-b show an embroidered conductor matrix on a knitted textile substrate. FIG. 32a shows a side of the garment facing skin, and FIG. 32b shows the opposite side of the garment.

FIG. 33 shows a carbon-based dry electrode fixed by thermal compression molding on to a textile substrate with a conductor positioned between the dry electrode and textile substrate (only the cable segment is shown).

FIG. 34 a-c show a textile-based functional electrical stimulation dry electrode garment sleeve for a forearm: FIG. 34a shows the forearm sleeve and zipper closure, FIG. 34b shows an outside surface of the forearm sleeve, and FIG. 34c shows the forearm sleeve connected to a system for delivering electrical stimulation to the dry electrodes in the forearm sleeve.

FIG. 35a-b show an experimental setup for using a dry electrode garment forearm sleeve during functional electrical stimulation FIG. 35a without stimulation (at rest) and FIG. 35b under stimulation (wrist extension).

### DETAILED DESCRIPTION

Currently available conventional transcutaneous stimulation electrodes (e.g., hydrogel) are often not reusable or washable and cannot be conveniently embedded in a wearable garment. Available dry electrodes (e.g., textile, carbon rubber, and metal) frequently cause pain to a wearer during electrical stimulation and require wetting or an intervening layer, e.g., gel layer, to render them comfortable during use. Available hydrogel electrodes are comfortable to use, but they dry out and degrade over time or repeated use, and therefore require frequent replacement.

Described herein is a completely dry transcutaneous electrode 2 for stimulation purposes which does not require a wet interface to operate effectively. The electrode is a thin film made from a polymer nanocomposite blend (e.g., PVDF+CNT) which is durable, flexible, and can for example have a smooth non-adhesive surface.

Conductive polymer composites are robust structures fabricated by the combination of thermoplastic polymers and conductive nano fillers providing unique properties with high mechanical flexibility and durability [4]. As set out in the Examples, when combined with appropriate components, they are electrically conductive, able to conform to the human body, and the inventors have discovered that they can be used as thin-film electrodes for different electrical stimulation applications [4]. The inventors have further discovered that the stable chemical structure of conductive polymer composites allows for rewashing and reuse without inducing changes to the composition. These features make electrodes fabricated with conductive polymer composite materials superior to conventional gel electrodes or those used with permanent conductive chemical adhesives that cannot be re-used after several hours of use.

As detailed in the Examples, the inventors have further found that the carbon-based dry electrode 2 can be integrated into a textile substrate 12 by a thermal compression molding process on an embroidered conductive matrix. This matrix is composed of textile silver-plated conductive yarns 10 and is linked to a stimulator 8. Besides ensuring the electrical connection, the matrix improves the fixation between the textile substrate 12 and the dry electrode 2. Stimulation intensity, perceived comfort and muscle torque generated by a smart FES sleeve 4 were compared to hydrogel electrodes.

The present disclosure relates to, in an aspect, reusable dry electrodes 2 comprising a conductive material made from conductive carbon nanoparticles dispersed in a fluoropolymer matrix that is non-reactive and/or chemically stable and a conductor element configured to contact the conductive material. When connected to a stimulator 8, electrical pulses delivered through the conductor 10 to the conductive material contacting tissue are directly and comfortably delivered to the underlying tissue without requiring wetting or having to pass through an intervening layer (e.g., an interface material or hydrogel).

The conductive material is for example non-metallic.

In another aspect, the present disclosure relates to reusable dry electrodes 2 incorporated into washable articles or garments 4 that can be worn and comfortably used with electrical stimulation without requiring wetting or use of an interface layer.

The present disclosure also in a further aspect relates to methods of manufacturing washable dry electrode articles or garments 4, which include fabricating the herein-disclosed reusable dry electrodes 2 and incorporating the dry electrodes 2 into an article or garment 4 to be worn and comfortably used with electrical stimulation without requiring wetting or use of an interface layer.

In yet a further aspect, the present disclosure relates to dry electrode devices assembled with dry electrode articles or garments 4 comprising reusable dry electrodes 2 and a stimulator 8 connected to the conductor elements contacting the conductive material of the dry electrodes 2. In some embodiments, connectors 6 linked to a portable stimulator 8 are also incorporated into the article or garment 4. In these embodiments, the portable stimulator 8 can be discreetly carried in or on the article or garment 4, for example, in a pocket.

In another aspect, the present disclosure relates to uses of reusable dry electrodes 2 and articles or garments 4 and/or devices comprising reusable dry electrodes 2, including those manufactured using the herein-described methods of manufacture, for treating mobility disorders in users in need of treatment. In some embodiments, mobility disorder treatments utilizing the herein-disclosed reusable dry electrodes 2 and articles or garments 4 and/or devices include positioning dry electrodes 2 or articles or garments 4 comprising same on tissue of a user in proximity to a selected muscle of group of muscles then delivering electrical stimulation through the conductor 10 to comfortably induce contraction of the muscle or muscles. In some embodiments, the mobility disorder treated is a form of paralysis.

### Definitions

As used herein, the term “Ω” means ohms.

As used herein, the term “Ω·cm” means ohm-centimeters.

As used herein, the term “Ω·m” means ohm-meters.

As used herein, the term “Ω/cm” means ohms per centimeter.

As used herein, the term “Ω/m” means ohms per meter.

As used herein, the term “μs” means microseconds.

As used herein, the term “μm” means micrometers.

As used herein, the term “GPa” means Gigapascals.

As used herein, the term “Hz” means hertz.

As used herein, the term “kHz” means kilohertz.

As used herein, the term “mA” means milliamperes.

As used herein, the term “mm” means millimeters.

As used herein, the term “ms” means milliseconds.

As used herein, the term “MPa” means Megapascals.

As used herein, the term “wt %” means weight percent.

As used herein, the term “aspect ratio” means length-to-diameter ratio.

As used herein “article” means any item that can make direct contact with and/or can conform to a body part of a user. Examples include non-wearable items that can make direct contact with a body part of a user, such as bedding, shoe insoles, rugs, mats, carpeting, and furniture coverings or upholstery that can be placed on, for example, chairs, seats, etc., and other similar such items and derivatives thereof.

The term “conductor” as used herein, and as is well understood in the art, means any material that is inherently or intrinsically capable of conducting electrical current. Examples include metallic materials such as silver-coated yarns, copper-coated yarns, yarns containing silver fibers, yarns containing copper fibers, carbon fibers and yarns, etc.

As used herein, the term “directly” and its derivatives mean passing from one point to another without interacting with an intervening component. For example, delivering electrical stimulation “directly” to a tissue from an electrode should be construed to mean that the stimulation is delivered to the tissue without first interacting with an intervening component, for example an interface (e.g., hydrogel), disposed between the electrode and the tissue.

As used herein, “disposing”, “disposing on”, “disposed”, “disposed on” and derivatives refers to any means of combining one component with another component such that the two components are reversibly or non-reversibly in contact with one another, including, for example, by affixing, attaching, embedding, coupling, joining, linking, associating, connecting, incorporating, integrating and/or merging, etc., of the two components.

As used herein, “dry” means substantially lacking in moisture, humidity or wetness that arises from sources other than a biological tissue. For example, a “dry” electrode should be construed to mean an electrode that substantially lacks moisture during use, such as moisture arising from soaking the electrode in water or from addition of a hydrogel interface layer, however it is not to be construed to mean an electrode that becomes moist or humid during use as a result of said use, for example, use that causes sweating in a user which leads to wetting of the electrode during use.

As used herein, the term “garment” means any item that is wearable on and/or conforms to and makes direct contact with a body part of a user. Examples include wearable items, such as clothing, shirts, pants, socks, wraps, cuffs, sleeves, gloves, boots, braces, suits, helmets, apparel and other similar such items and derivatives thereof.

As used herein, and as is well understood in the art, the term “impedance” means the amount of impedance (i.e., combined resistance and capacitance) a component offers to current flowing through a circuit at a specific frequency as measured in ohms (Ω).

As used herein, the term “improved comfort” means an increased level of perceived comfort (e.g. of one or more parameters as reported by a user) of an electrode as compared to perceived comfort levels during use of a different type of electrode, e.g., a conventional electrode known in the art (e.g., sponge, carbon rubber, hydrogel and textile transcutaneous electrodes). For example, perceived comfort levels of an electrode user can be evaluated with a comfort assessment tool known in the art, such as obtaining verbal reports of how comfortable the stimulation felt using a visual numerical rating scale from 0 to 10 displayed to the user, wherein a comfort level of 0 indicates “very comfortable” and a comfort level of 10 indicates “very uncomfortable”. In this context, “improved comfort” should be construed to mean relatively higher levels of reported comfort during use of an electrode with electrical stimulation compared to comfort levels reported during use of a different type of electrode with electrical stimulation delivered by the same type of stimulator using consistent stimulation pulse parameters (e.g., a uniform pulse frequency and pulse width). For example, “improved comfort” can indicate relatively higher levels of reported comfort (e.g., a lower average comfort level score on one or more parameters) associated with use of a herein-disclosed dry polymer nanocomposite electrode used with a particular pulse, for example a pulse having a frequency of 40 Hz and width of 300 μs delivered at high or maximum tolerated intensities by a particular stimulator, for example a MyndSearch stimulator, compared to reported comfort levels associated with use of a conventional hydrogel electrode used with a similar pulse, e.g having a frequency of 40 Hz and width of 300 μs delivered at high or maximum tolerated intensities by a MyndSearch stimulator. The improved comfort can be in the context of comparing a dry polymer nanocomposite electrode as disclosed herein to a different electrode where the comparison is made without an interface layer, such as a hydrogel or wetting.

As used herein, “interconnected” means individual components making physical contact with one another. For example, interconnected nanoparticle units in a polymer matrix make contact with one another, for example because they are physically entangled, to create a conductive network of nanoparticle units.

As used herein, “mobility impairment” means any disability or condition which limits motor ability in any body area or part. Examples of disabilities or conditions that limit motor ability include, but are not limited to, congenital anomalies such as, for example, spina bifida, central nervous system injuries resulting from, for example, head trauma, spinal cord injuries resulting from, for example, physical trauma or accidents, paralysis or partial paralysis, injuries resulting from disease such as, for example, muscular dystrophy, multiple sclerosis, cerebral palsy, stroke and brachial plexus injuries.

As used herein, “portable” means capable of being carried, moved or transported.

As used herein, “reusable” means capable of being re-used multiple times (e.g., more than 2-3 times) without a significant change in physical integrity and/or performance of an item, under normal use conditions. For example, a reusable dry electrode as herein described may be re-used more than 2-3 times, e.g., 10, 100 or 1000 times, wherein the dry electrode provides substantially consistent stimulation to a user each time it is used (e.g., it provides consistent muscle torque when used with a stimulator delivering pulses having substantially similar characteristics).

As used herein, “rise time” with respect to a pulse refers to the time required for the pulse to go from an initial stimulation intensity level (e.g., 0 mA) to a final stimulation intensity level (e.g., 25 mA) within a single pulse.

As used herein, “linear resistance” refers to a measure of how much a conductive component resists the flow of electrical current as measured in ohms per centimeter (Ω/cm) or ohms per meter (μ/m).

As used herein, “sheet resistivity” refers to a measure of how much a component resists the flow of electrical current in a thin sheet or film as measured in ohm-centimeters (Q·cm) or ohm-meters (Ω·m) and can be used to find the electrical conductance of a film component.

As used herein, “tissue” means any biological tissue capable of receiving electrical stimulation. Examples include skin, skeletal muscle, cardiac muscle, smooth muscle and nervous system tissues such as brain, spinal cord, peripheral nerves, auditory or optical nerves.

As used herein, “washable” means a component capable of being repeatedly washed, re-washed or cleaned without damaging or otherwise compromising or impairing the physical integrity and/or functionality of the component over a defined period time.

As used in this specification including the appended claims, the singular forms “a”, “an” and “the” include the plural references unless the content clearly indicates otherwise. Thus, for example, “an electrode” includes two or more electrodes.

As used in this specification including the appended claims, the term “or” is generally employed in its sense including “and/or” unless the content clearly indicates otherwise.

As used herein, the terms “about”, “substantially” and “approximately” mean a reasonable amount of deviation of the modified term such that the end result is not significantly changed. This term of deviation should be construed to include a deviation of at least ±5%, or at least ±10% of the modified term if this deviation would not negate the meaning of the word it modifies.

As used in this specification including the appended claims, the word “comprises” and its derivatives such as “including” and “having” are intended to be open-ended terms that specify that the stated features, elements, components, groups, integers, and/or steps do not exclude the presence of other unstated features, elements, components, groups, integers and/or steps.

The definitions and embodiments described in particular sections are intended to be applicable to other embodiments herein described for which they are suitable as would be understood by a person skilled in the art. For example, in the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Dry Electrodes

A first aspect provided herein relates to a reusable and transcutaneous dry electrode 2 comprising i) a conductive material further comprising conductive carbon nanoparticles dispersed in a fluoropolymer matrix, and ii) a conductor 10 configured to contact and deliver electrical pulses to the conductive material from a stimulator. The conductive material can be, for example, non-metallic. A dry surface of the conductive material is configured to make contact with a tissue such to comfortably deliver transcutaneous electrical pulses directly to the tissue without any wetting of the electrode 2 or use of an intervening layer configured to interface between the electrode and the tissue (e.g., hydrogel). The disclosed reusable dry electrode 2 can provide comparable or improved comfort to a user compared to conventional electrodes known in the art during similar electrical stimulation conditions (e.g., at high or maximum stimulation intensities).

The herein-disclosed dry electrode 2 is configured, for example, to deliver rather than receive electrical stimulation.

A reusable transcutaneous dry electrode 2 as herein described may have a generally smooth surface (e.g., having even and consistent surface features) and/or a non-adhesive surface (e.g., a surface that does not adhere or stick to other surfaces or objects), and does not require surface modification (e.g., physical or chemical surface modification) for effective delivery of transcutaneous electrical pulses to a tissue.

By nature of the composition of the conductive material, reusable dry electrodes 2 as herein described are generally robust or long-lasting, durable, and highly mechanically flexible such to contour a human body part, e.g., a limb, and sterilizable. In some embodiments, sterilization of a dry electrode 2 as herein described can be carried out using known methods, for example, exposing the material to compatible sterilizing chemicals or gases, radiation, high temperatures (e.g., as high as 125 to 350° C.) or steam under high pressures and/or high temperatures (e.g., as high as 125 to 350° C.). In preferred embodiments, the herein-disclosed dry electrodes 2 can be used with electrical stimulation or functional electrical stimulation (FES) for at least 10 hours or more while maintaining its electrical and mechanical properties.

Reusable dry electrodes 2 as herein described can be comfortably worn by a user for an extended period of time (e.g., 10 hours or more, or 16-24 hours) as they do not require use with a wetting agent (e.g., water) or hydrogel and are thermally stable.

In some embodiments, the dry electrode 2 is an extended use electrode which can be comfortably worn for an extended period of time (e.g., 10 hours or more, or 16-24 hours).

In preferred embodiments, the fluoropolymer matrix is polyvinylidene fluoride (PVDF).

In some embodiments, the conductive nanoparticles can include carbon black, graphene nanoplatelets or single-walled carbon nanotubes (CNTs). In preferred embodiments, the conductive nanoparticles are multi-walled CNTs, which are commercially available from, for example, Nanocyl or Sigma-Aldrich. In some embodiments, the conductive nanoparticles are preferably dispersed in the substrate at about 5 to 10 wt % of the substrate. In some embodiments, the nanoparticles have an aspect ratio of about 1 to 10,000. In other embodiments, the nanoparticles have an aspect ratio of about 1 to 600. In preferred embodiments, the nanoparticles have an aspect ratio of about 130 to 160. In some embodiments, the nanoparticles are interconnected. For example, multi-walled carbon nanotubes (CNT-NC7000) from Nanocyl™ (Belgium), cPD15L1-5 multiwall carbon nanotubes-hollow structure, from NanoLab™, or graphene nanotubes (single wall carbon nanotubes) from Tuball™ can be used.

In some embodiments, the stiffness represented by the elastic modulus of the conductive material is around 1.2 to 0.8 Gigapascals (GPa). In other preferred embodiments, the strength of the conductive material presented by the yield strength is around 16 to 10 Megapascals (MPa).

In some embodiments, the conductive material has an impedance of about 10 to 100,000Ω. In other embodiments, the conductive material has an impedance of about 100 to 10,000Ω. In other embodiments, the conductive material has an impedance of about 1,000 to 10,000Ω. In yet other embodiments, the conductive material has an impedance of about 1,000 to 5,000Ω. In preferred embodiments, the conductive material has an impedance of about 2,100 to 2,800Ω.

In some embodiments, the conductive material has a sheet resistivity of 50 to 500 Ω·cm. In preferred embodiments, the conductive material has a sheet resistivity of 100 to 300 Ω·cm.

In preferred embodiments, the conductive material has an impedance of about 1,000 to about 10,000Ω and a sheet resistivity of about 50 to about 500 Ω·cm.

In preferred embodiments, the conductive material has an impedance of about 2,100 to about 2,800Ω and a sheet resistivity of about 100 to about 300 Ω·cm.

In some embodiments, the conductive material is formed as a film-like layer and/or has a width of about 50 to 200 μm. In preferred embodiments, the conductive material has a width of about 50 to 100 μm.

In some embodiments, the tissue is human tissue. In preferred embodiments, the tissue is skin. In other preferred embodiments, the tissue is human skin.

In some embodiments, the dry electrode 2 is adapted for positioning on the skin in proximity to a selected muscle or group thereof. In preferred embodiments, the electrical stimulation induces a contraction of the muscle or group thereof.

In some embodiments, the dry electrode 2 is used with electrical stimulation. In preferred embodiments, the electrical stimulation used or to be used with the dry electrode 2 as herein described is functional electrical stimulation.

In some embodiments, the dry electrode 2 as herein described provides an improved comfort to a user. For example, as demonstrated herein, at high or maximum stimulation intensities, a dry electrode 2 as herein described used with a high-rise voltage-regulated stimulator 8 such as a MyndSearch stimulator, yields improved comfort to a user, for example, a reported total average comfort rating of approximately 1 to 6 on a self-reported comfort scale of 1 to 10 (with 1 being the most comfortable), compared to a conventional electrode used with a MyndSearch stimulator using substantially similar stimulation parameters. As shown herein, higher stimulation intensities can be comfortably used with a herein-described dry electrode 2 with EV-906 and MyndSearch stimulators. For example, up to approximately 20% higher stimulation intensity can be comfortably used with a herein-described dry electrode and an EV-906 or MyndSearch stimulator, as compared to use of the dry electrode with a Compex stimulator.

In some embodiments, the electrode 2 is disposed on or incorporated or integrated into a dry electrode article or garment 4, which can be wearable or non-wearable. In other embodiments, the electrode 2 is integrated into, or is made integral with, an article or garment, for example by casting the electrode 2 directly on to a surface of the garment using, for example, compression-molding. In other embodiments, the garment is an everyday wearable garment. In preferred embodiments, the electrode 2 is disposed on an inside surface of the article or garment 4 such to juxtapose the dry tissue-contacting surface of the electrode 2 against the tissue. In some embodiments, the electrode 2 is disposed on or attached or bound to the article or garment 4 using, for example, sewing, stitching, embroidery, and other known means of attachment or binding. In embodiments, the electrode 2 is affixed to the article or garment 4 with an adhesive. In other embodiments, a suitable adhesive used with the invention is of a type known within the art and suitable for use with textiles, for example, glue, fabric glue, bonding tape, rivets, and the like. In preferred embodiments, as described further below, the dry electrode 2 is integrated into the article or garment 4 using thermal compression.

### Dry Electrode Articles or Garments and Methods of Manufacture

Another aspect relates to washable dry electrode articles or garments 4 comprising a reusable dry electrode 2 as herein described.

In some embodiments, the dry electrode can be disposed on, or incorporated or integrated into, articles or garments 4, for example everyday garments, to facilitate the delivery of electrical stimulation or functional electrical stimulation (FES) therapies to a broad patient population outside of clinical settings.

In some embodiments, the article or garment 4 comprises the dry electrode 2, for example, as an integrated layer, or as a layer disposed on an inside surface of the article or garment such to juxtapose the tissue-contacting surface of the conductive material against the tissue.

In some embodiments, the article or garment 4 and dry electrode 2 are attached using, for example, binding, sewing, stitching, embroidery, compression-molding and other known means of combination or attachment. In other embodiments, the article or garment 4 is affixed to the dry electrode 2 with an adhesive. In other embodiments, a suitable adhesive used with the invention is of a type known within the art and suitable for use with textiles, for example, glue, fabric glue, bonding tape, rivets, and the like.

In other embodiments, the conductor 10 comprises an electrode-contacting segment 14 which makes contact with the dry electrode 2 disposed on the article or garment 4 textile substrate 12. Within the electrode-contacting segment 14, the conductor 10 is integrated into the textile substrate 12. In these embodiments, the conductor 10 may be or comprise one or more conductive yarns 100. The conductive yarns 100 can be integrated into the textile substrate 12 (and/or any other layer such as a thermoadhesive) via any means known in the art, for example, sewing, embroidery, knitting or weaving.

In embodiments, the article or garment 4 is wearable and formed with one layer of material made from a non-conductive knitted textile substrate 12, for example, a knitted compression fabric, which is flexible and adaptable to the shape of a body part such as arms, legs, torso, etc., and can make close contact with skin. Examples of wearable dry electrode articles or garments 4 can include a face mask, a neck collar, a glove, a shirt, a sleeve, pants, etc, comprising a dry electrode 2 and which can be worn on and stimulate the muscles of any part of the body, for example, pectoral, deltoid, trapezius muscles. In preferred embodiments, the textile substrate is or can comprise polyester jersey fabric, for example, PET (polyethylene terephthalate) jersey.

In embodiments, portions of the conductive yarns 100 are integrated into the textile substrate 12 in the electrode-contacting segment to form a pattern comprising a plurality of lines which do not intersect with each other, and which make contact with the dry electrode 2 disposed on the article or garment 4. In these embodiments, each line is formed by one conductive yarn 100. If the lines made of separate conductive yarns 100 are integrated such to intersect or make contact with each other, stimulation delivery to the dry electrode 2 disposed on the textile substrate may be less effective due to short circuiting among the individual conductive yarns 10. Lengths of at least two of the plurality of non-intersecting lines can be arranged substantially parallel relative to each other within the electrode-contacting segment 14, or lengths of the non-intersecting lines can be substantially non-parallel or splayed relative to each other within the electrode-contacting segment 14 to form, for example, a fan pattern having a center node aligned approximately where a non-integrated cable segment 16 of the conductor begins, which comprises converging and non-integrated portions of the conductive yarns 100.

In an embodiment, the electrode-contacting segment 14 comprises more than two non-intersecting lines that are substantially parallel relative to one another.

In an embodiment, the electrode-contacting segment 14 comprises seven (7) lines that are substantially parallel relative to one another.

In embodiments, the integration pattern of the conductor electrode-contacting segment 14 can be formed to resemble any shape that maintains the lines in non-intersecting tracts, for example, a series of non-intersecting zig zag-, wave-, or spiral-shaped tracts. Any such integration pattern can be used with dry electrodes 2 of any desired shape (e.g., square, round, rectangular, etc.).

In preferred embodiments, the lines form a pattern which resembles a candelabrum, wherein at least one center line runs in a straight line toward the cable segment and divides the electrode-contacting segment 14 into two approximately equal areas. The remaining non-center lines run substantially parallel to each other and the center line in the portion of the electrode-contacting segment opposite to the cable segment 16, but take an approximately 900 turn in a portion proximal to the cable segment 16 such to run perpendicular and toward the center line without intersecting one another (e.g., they continue to run approximately parallel to each other). The center line positioned at the center of the electrode-contacting segment thus forms a dividing center line wherein approximately mirror image and differently sized “L” and “J” tracts are nested relative to each other and on either side of the dividing center line.

In embodiments, the lines can be integrated into the textile substrate 12 such to cover an area substantially corresponding to the shape of the dry electrode 2 disposed on the textile substrate 12, for example, a circular or square shape.

In other embodiments, the conductor 10 further comprises a cable segment 16. In embodiments wherein the conductor is or comprises one or more conductive yarns 100, the cable segment 16 comprises non-integrated portions of the conductive yarns 100. The non-integrated portions are configured for delivering electrical stimulation to the conductor electrode-contacting segment 14, for example by contacting a connector 6 linking the conductor 10 to a stimulator 8. The conductor cable segment 16 can be secured to the textile substrate 12 by any attachment means known in the art, for example, by sewing the cable segment to the textile substrate 12 using non-conductive thread, by gluing the cable segment to the textile substrate 12, or by using fasteners to tether the cable segment to the textile substrate 12.

In embodiments, the conductor cable segment 16 comprises more than one conductive yarn 100 crimped, woven or otherwise fastened together.

In embodiments, the conductive yarns 100 are or comprise silver-plated yarns. Any stimulator 8 known in the art can be connected to the conductive yarns 100 to deliver stimulation thereto. In preferred embodiments, a MyndSearch stimulator is connected to the conductive yarns 100 via connectors 6 known in the art, for example pluggable/unpluggable connectors.

In embodiments, the conductive yarns 100 have a linear resistance of at least 1 Ω/m or about 1 to about 1000 Ω/m.

In other embodiments, a protective layer 20 is disposed on a non-skin-contacting surface of the article or garment 4 to cover exposed portions of the conductive yarns 100. In these embodiments, the protective layer 20 shields any exposed portions of the conductive yarns 100 from contacting other conductive objects, which could lead to short circuiting and less effective stimulation delivery to the dry electrode 2 disposed on the article or garment 4. The protective layer 20 can also provide protection to the conductive parts of a dry electrode article or garment 4 from mechanical and chemical stresses experienced during washing cycles. The protective layer 20 can be, for example, a fabric layer attached to the article or garment 4 such to cover the dry electrode 2 disposed on the article or garment 4, a liquid silicone layer spread over the dry electrode 2, or a covering embroidery or polyurethane film.

Wearable dry electrode articles or garments 4 as herein disclosed may be simpler to use for patients and healthcare professionals while being at least as comfortable, if not more comfortable, for patients compared to other wearable stimulation devices known in the art which incorporate, for example, hydrogel electrodes known in the art. Further, the herein-disclosed dry electrode articles or garments 4 do not require use of a gel layer such as required with, for example, hydrogel electrodes, which can lead to discomfort and cause the hydrogel electrode to shift during use.

Another aspect relates to methods of manufacturing washable dry electrode articles or garments 4, which comprise fabricating a dry electrode 2 as herein described by i) dispersing conductive carbon nanoparticles into a polymer solvent to yield a dispersion, ii) dissolving fluoropolymer matrix in the dispersion to yield a blend, iii) casting the blend, v) drying the casted blend, vi) compressing the blend, vii) cooling the blend to yield a conductive material, viii) cleaning the material, ix) disposing a conductor 10 on the material, wherein the conductor 10 is configured to contact and deliver an electrical pulse to the material from a stimulator 8, and further comprising incorporating the manufactured dry electrode of into an article or garment, wherein the electrode 2 is disposed on an inside surface of the article or garment such to juxtapose the tissue-contacting surface of the electrode against the tissue for delivering electrical stimulation directly thereto, for example, without passing through an intervening layer disposed between the tissue-contacting surface and the tissue.

In preferred embodiments, the casting and/or compressing comprises transferring the blend into a mold or dish. Casting molds or dishes used to shape the conductive material may be made from durable materials known in the art, such as aluminum or steel, and can be custom-made to shape the conductive material into any desired shape, for example, any shape or size based on standard commercial electrode dimensions used with electrical stimulation or functional electrical stimulation (FES) applications, or circular, square and/or rectangular shapes. In other embodiments, the conductive material is custom-shaped to contour a particular body part, or to contour a particular body part of an individual. In some embodiments, the conductive material is shaped into a circular form having a diameter of 25 mm and/or a square form having a side length of 50 mm.

In preferred embodiments of the methods of manufacturing as disclosed herein, the polymer solvent is dimethylformamide (DMF).

In preferred embodiments, the dispersing is effected by sonication.

In some embodiments, the dissolving, casting and/or drying further comprises heating the blend. In preferred embodiments, the heating is effected within about 80 to 220° C.

In some embodiments, the compressing is effected using a commercially available molding press system such as the Bench Top Auto Press available from Carver, Inc. In preferred embodiments, the applied compression force is about 5 to 10 tons.

In some embodiments, the cleaning is effected with water and/or ethanol.

In other embodiments, the disposing a conductor 10 on the material according to (viii) above comprises integrating at least one conductive yarn 100 through a knitted textile substrate 12, such as a textile substrate 12 configured to fit over a body part. The integrating can be carried out using any method known in the art, such as sewing or embroidering.

In other embodiments, disposing the dry electrode 2 according to (B) above further comprises i) positioning a thermoadhesive 18 on the textile substrate 12, and then integrating one or more conductive yarns 100 through both the textile substrate 12 and thermoadhesive 18, ii) positioning the dry electrode 2 on the thermoadhesive, and iii) affixing the thermoadhesive to the dry electrode 2 and textile substrate 12 using thermal compression. Thermal compression applies sufficient heat to melt at least a portion of the conductive material of the dry electrode 2 while simultaneously applying pressure such to contact and integrate at least a portion of the one or more conductive yarns 100 into the melted portion of the dry electrode 2 material. Thermoadhesive 18 is made of a woven or non-woven substrate or fabric configured to be placed between the dry electrode 2 and the textile substrate 12. Thermal compression may melt a portion of the thermoadhesive material to create adhesion between layers Thermal compression should be carried out at a temperature sufficient to melt the dry electrode 2 material, for example, at least 180° C. or between about 180° C. and 230° C., and at pressures sufficient to integrate the conductive yarns 100 into the melted dry electrode 2 material. For example, pressures of about 1 to 10 tons can be applied for a time, for example, between the range of 5 to 10 minutes, such as about 1 ton of pressure applied to the dry electrode 2 and textile substrate 12 for at least 5 minutes. This can be followed by applying about 5 tons of pressure to the melted dry electrode 2 material and textile substrate 12 for at least about 5 minutes, or up to about 7 minutes.

In preferred embodiments, the thermoadhesive 18 material is made of a non-woven material configured to maximize adhesion between the dry electrode 2 and the textile substrate 12, for example, a non-woven thermoadhesive material providing adhesion under heat and compression to the dry electrode 2.

### Dry Electrode Devices and Methods of Use

Another aspect relates to a dry electrode device for delivering electrical stimulation to a subject comprising i) a dry electrode article or garment 4 as herein described, ii) a stimulator 8, and iii) a connector 6 linking a conductor 10 or 100 as herein described and the stimulator 8.

In some embodiments, the dry electrode 2 is integrated into or is made integral with an article or garment, for example by casting the electrode 2 directly on to a surface of the article or garment using, for example, compression-molding. In preferred embodiments, the garment is an everyday garment. In some embodiments, the electrode 2 is disposed on an inside surface of the article or garment such to juxtapose the dry tissue-contacting surface of the electrode against tissue of a user. In some embodiments, the electrode 2 is attached to the article or garment using, for example, binding, sewing, stitching, embroidery, and other known means of attachment. In some embodiments, the electrode 2 is affixed to the article or garment with an adhesive. In other embodiments, a suitable adhesive used with the invention is of a type known within the art and suitable for use with textiles, for example, glue, fabric glue, bonding tape, rivets, and the like. In preferred embodiments, the dry electrode 2 is integrated into a dry electrode article or garment 4.

A herein-described dry electrode 2 is lightweight compared to conventional electrodes known in the art (e.g., carbon rubber electrodes) and is therefore well-suited for use in an article or garment. When integrated into an article or garment 4, the dry electrode 2 minimally impacts the shape thereof and can contribute to providing increased comfort to a wearer thereof compared to garments comprising conventional electrodes.

In some embodiments, a connector 6 linking the conductor 10 or 100 and stimulator 8 is of a type known within the art, for example, copper wire, silver-coated yarns, and carbon fibers and yarns. In some embodiments, the connector 6 is disposed on the article or garment 4. In preferred embodiments, the connector 6 is incorporated or integrated into the article or garment 4.

In some embodiments, the stimulator 8 is non-portable. In preferred embodiments, the stimulator 8 is portable, rechargeable and/or battery-operated. In further preferred embodiments, the battery-operated stimulator 8 functions without battery replacement or having to be recharged for at least 10 hours. In other preferred embodiments, the portable stimulator 8 is configured to be positioned within a portion of the article or garment 4, for example, a pocket disposed on the garment.

In another aspect, a dry electrode article or garment 4 and/or dry electrode device as herein described and/or a dry electrode 2 manufactured by a method of manufacture as herein described is used for treating a mobility impairment in a user.

In another aspect, a dry electrode 2 as herein described is manufactured by a method of manufacture as herein described and/or is used for treating a mobility impairment, for example paralysis, in a user.

In some embodiments, a dry electrode article or garment 4 and/or dry electrode device as herein described is used for treating a mobility impairment, for example paralysis, in a user.

In another aspect, a dry electrode article or garment 2 and/or dry electrode device as herein described and/or a dry electrode 2 manufactured by a method of manufacture as herein described is used for muscle strengthening and muscle relaxation and massage-like interventions in athletes, healthy individuals as well as in individuals with various types of neuromuscular impairment.

In another aspect, a dry electrode article or garment 4 and/or dry electrode device as herein described and/or a dry electrode 2 manufactured by a method of manufacture as herein described is used to treat pain using electrical stimulation-based technology, for example transcutaneous electrical nerve stimulation (TENS) or other similar technology.

In another aspect, a dry electrode article or garment 2 and/or dry electrode device as herein described and/or a dry electrode manufactured by a method of manufacture as herein described is used to provide sensory stimulation to patients with impaired sensory function, for example, amputees and individuals that have damaged peripheral nervous systems.

In preferred embodiments, uses of a dry electrode 2 or dry electrode article or garment 4 as herein described comprise i) positioning a dry electrode article or garment 4 or at least two dry electrodes 2 as herein described directly on the tissue of a user in proximity to a selected muscle or group thereof, ii) connecting the conductors 10 or 100 of the article or garment or of the at least two dry electrodes 2 to the stimulator, and iii) delivering electrical stimulation from the stimulator 8 to the conductors 10 or 100 of the article or garment 4 or the at least two dry electrodes 2, wherein the stimulation induces a contraction of the muscle or group of muscles to treat a mobility impairment.

In some embodiments, uses in treatment of a mobility impairment disorder comprise electrically stimulating or inducing muscle contractions such to cause movement generation in a user, for example, standing up, walking, grasping an object, or making a facial expression. In other embodiments, specific or selected sequences of electrical stimulation generate muscle contractions and generate movements, for example, by varying the timing of electrical stimulation delivered to particular muscles, or by simultaneously delivering electrical stimulation to selected sets of muscles through use of multiple dry electrodes 2.

In another aspect, methods of delivering electrical stimulation to a subject using a dry electrode 2 or dry electrode article or garment 4 as herein described comprise i) positioning an article or garment 4 as herein described or at least two dry electrodes 2 as herein described directly on tissue of a subject in proximity to a selected muscle or group of muscles, ii) connecting the conductors 10 or 100 of the article, garment 4 or dry electrodes 2 to a high-rise voltage-regulated stimulator 8, and iii) delivering an electrical pulse from the stimulator 8 to the conductors 10 or 100 of the article or garment 4 or dry electrodes 2, wherein the stimulation induces a contraction of the muscle or group of muscles.

In some embodiments, the dry electrode 2 is configured for use with a high-rise voltage-regulated stimulator 8, wherein the electrode 2 is configured to deliver an electrical pulse, for example, a monophasic or biphasic electrical pulse. In some embodiments, the pulse is monopolar. In other embodiments, the pulse is bipolar. In preferred embodiments, suitable stimulators 8 for use with the herein-described dry electrodes 2 are high-rise voltage-regulated stimulators, for example, EV-906 (Everyway Medical Instruments, Taiwan) and MyndSearch stimulator (MyndTec, Canada). In preferred embodiments, the dry electrode 2 is used or is for use with a stimulator 8 capable of delivering a pulse having a rise time of less than 40 ns or, for example, about 10 to 40 ns, for example, a MyndSearch stimulator (MyndTec, Canada). In other preferred embodiments, the dry electrode 2 is used with a functional electrical stimulation system or device as described in U.S. Pat. Nos. 8,880,178 and 9,440,077.

In some embodiments, the pulse the dry electrode 2 is configured for use with has a total width (i.e., from beginning of the pulse to the end of the pulse) of about 8 to 2,000 μs. In other embodiments, the pulse has a total width of about 10 to 1,500 μs. In yet other embodiments, the pulse has a total width of about 100 to 1,000 μs. In preferred embodiments, the pulse total width is about 200 to 500 μs.

In some embodiments, the pulse is delivered at a frequency of about 1 to 100 Hz. In other embodiments, the pulse is delivered at a frequency of about 16 to 100 Hz. In preferred embodiments, the pulse is delivered at a frequency of about 20 to 40 Hz. In other preferred embodiments, the pulse has an amplitude of about 0.5 to 120 mA.

In some embodiments, the pulse is balanced and/or symmetric. In preferred embodiments, the pulse is biphasic and/or symmetric or asymmetric and comprises a first phase pulse and a second phase pulse. In preferred embodiments, the rise time of the pulse is about 10 to about 40 ns. In some embodiments, the first phase pulse has an amplitude of about −0.04 to −160 mA. In preferred embodiments, the first phase pulse has an amplitude of about −0.4 to −120 mA and/or a width of about 200 to 500 μs. In some embodiments, the second phase pulse has an amplitude of about 0.1 to 30 mA. In other embodiments, the second phase pulse has an amplitude of about 0.4 to about 120 mA and/or a width of about 200 to about 500 μs. In preferred embodiments, the second phase pulse has an amplitude of about 0.01 to 40 mA and/or a width of about 800 to 2,000 μs.

In some embodiments, the pulse is symmetric and/or the amplitude of the first phase pulse and amplitude of the second phase pulse are equal and/or the width of the first phase pulse and width of the second phase pulse are equal. In other embodiments, the first phase pulse amplitude is about −0.04 to about −160 mA and the second phase pulse amplitude is about 0.04 to about 160 mA. In other embodiments, the first phase pulse amplitude is about −0.4 to about −120 mA and the second phase pulse amplitude is about 0.4 to about 120 mA. In still other embodiments, the first phase pulse width is about 200 to about 500 μs and the second phase pulse width is about 200 to about 500 μs.

In preferred embodiments, the pulse is asymmetric and/or the amplitude of the first phase pulse and amplitude of the second phase pulse are unequal and/or the width of the first phase pulse and width of the second phase pulse are unequal. In preferred embodiments, the first phase pulse has an amplitude of about −0.4 to −120 mA and/or a width of about 200-500 μs and the second phase pulse has an amplitude of about 0.1 to 30 mA and/or a width of about 800 to 2,000 μs.

Dry electrode articles or garments 4, and/or devices as herein described can be used with any wireless communication means and/or any brain-controlled neuromodulation means known in the art to control stimulator 8 input to dry electrodes 2.

The following non-limiting examples indicate preferred embodiments of the invention and are merely illustrative of the presently disclosed invention.

### EXAMPLES

### Example 1: Dry Electrode Conductive Material

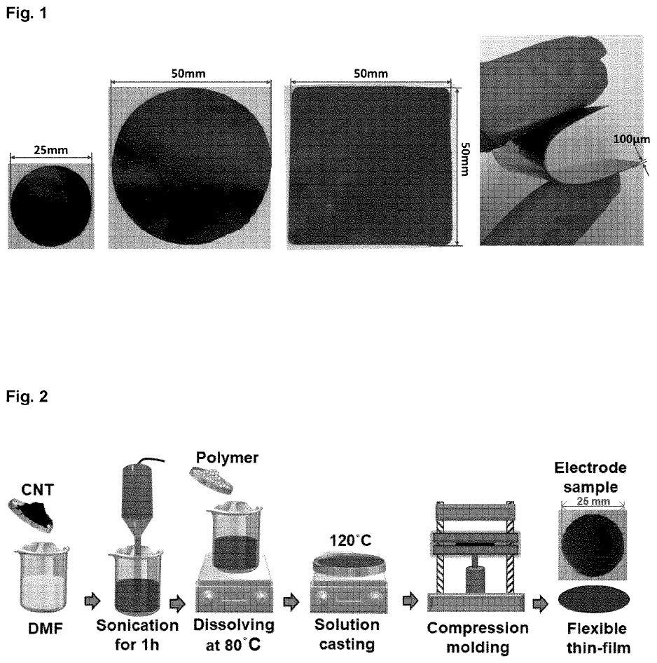

As shown in FIG. 1, the dry electrodes in this and the following non-limiting examples of the invention as herein-described were made with a flexible thin-film conductive polymer composite material. Use of PVDF as a polymer matrix and CNT as a conductive filler has shown to provide suitable electrical and mechanical properties to manufacture conductive thermoplastic polymer composites having the features needed for stimulation delivery [12], [13]. Use of PVDF as a polymer matrix and CNT as a conductive filler provided suitable electrical and mechanical properties for the polymer composite [6]. High electrical conductivity and uniform dispersion of CNT into the PVDF polymer matrix provided the desired conductivity of the polymer composite, and the biocompatibility, robustness, chemical stability of the selected polymer as well as full entanglement of the CNT into the matrix to deliver a durable, flexible, and washable thin-film layer for the electrode design to act as an interface between the skin and the stimulator connections.

The selected conductive nano-additive was Multi-walled Carbon Nanotube (CNT-NC7000) which was obtained from Nanocyl (Belgium) where the carbon nanotubes have an average diameter of 9.5 nm, average length of 1.5 μm, surface area of 25-300 m²/g, and volume resistivity of 10-4 Ω·cm on powder. The selected polymer solvent was Dimethylformamide (DMF) which was purchased from Sigma-Aldrich (USA). The main polymer matrix was made of Polyvinylidene Fluoride (PVDF) polymer where the PVDF pellets (Kynar™ 740) were supplied by Arkema (Canada). Ethanol was purchased from University of Toronto Chem Stores (Canada) for washing the samples. Aluminum molds for shaping the dry electrodes where machined at the Department of Mechanical and Industrial Engineering machine shop at the University of Toronto. All materials were used without any further purification. Deionized (DI) water was used for all experiments.

### Example 2: Manufacture of Dry Electrodes Using Solution Casting Process and Assembly Of Dry Electrodes

As illustrated in FIG. 2, a solution casting process was used to fabricate conductive polymer composite dry electrodes as described in Example 1 [12], [13]. First, the conductive filler (i.e., CNT at 5-10 wt % of the polymer) was dispersed into the polymer solvent (i.e., DMF at 93 wt % of the total solution) using a sonication process for 1 hour at 60 Watts. Then, the selected thermoplastic polymer pellets (i.e., PVDF) were added into the dispersion and mixed for 4 hours using a magnetic stirrer at 80° C. After full dissolution of the polymer, the solution was casted into a petri dish and dried on a hot plate for 12 hours at 120° C. The resulting dry PVDF-CNT blend was then transferred into a compression mold setup and heated at 180° C. to 220° C. for 6 minutes and then compressed into custom-made aluminum or steel molds for 3 to 5 minutes to obtain thin-film layers (50-100 μm) with the desired dimensions including circular or square shapes. The circular electrode had a diameter of 25 mm and the square shaped electrode had a side length of 50 mm. Both shapes and corresponding sizes were based on standard commercial electrode dimensions used for FES applications. The samples were then cooled down in a water bath for 5 minutes, and then removed from the mold. Finally, the resultant electrode conductive material was washed with water and ethanol, respectively, to yield dry electrodes ready for stimulation applications.

The dry electrode produced as described above was then attached to a conductive cable or line. The opposite side of the cable or line was attached to a connector that was directly plugged into the electrical stimulator or into a cable system that is commonly used with the stimulator to connect with conventional hydrogel electrodes.

### Example 3: Thermal and Mechanical Characterization of Dry Electrodes

As the dry reusable transcutaneous electrodes may be used in wearable applications, they may become exposed to high temperature such as a clothes dryer or potentially ironing. Therefore, the material's thermal stability behaviour at elevated temperatures was studied.

To investigate the dry electrode material properties and effect of the conductive nano filler (i.e., CNT), the thermal and mechanical properties of the polymer nanocomposite electrode material were examined and compared to the pristine PVDF polymer with no additives. The thermal degradation behavior of the polymer composite and pristine polymer were tested by thermal Thermogravimetric Analyses (TGA, Q50, TA Instruments, USA). The mechanical properties of the dry electrode and pristine polymer film samples were tested and compared with each other to show the mechanical stiffness and strength of the developed electrode. For this purpose, the tensile stress-strain behavior of three film samples was obtained by means of a Dynamic Mechanical Analyzer (DMA, Q800, TA Instruments, USA) using a force rate of 1 N/min.

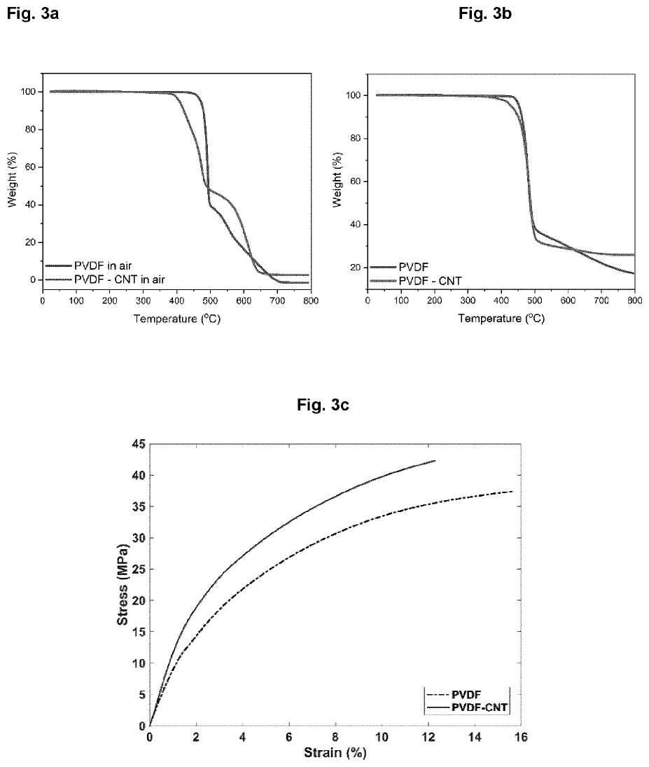

Thermal behavior of the polymer nanocomposite and pristine polymer, using Thermogravimetric Analyses (TGA) under air environment, are shown in FIG. 3(a). The initial degradation temperatures of the PVDF samples were around 436±2° C. and 383±2° C. for the pristine polymer and CNT nanocomposite, respectively. Both samples were thermally stable up to 350° C., which imply the electrode is safe for a day-to-day usage from the perspective of thermal degradation and indicates these electrodes could be embedded into garments and potentially endure the temperatures of washing, drying, and ironing processes. Samples showed relatively high initial degradation temperatures and the slight difference could be attributed to the dispersion of the CNT in the polymer matrix, similar to the results reported by [14]. According to non-limiting theory, the reduction in the onset temperature is hypothesized to be due to the CNT acting as a defect between the polymer matrix and the interface sites initiating the degradation, therefore reducing the initial onset degradation temperature [14]. Above 700° C., all samples were fully degraded as the final weight was close to 0%. For comparison, the thermal stability results from standard TGA test under nitrogen environment is also shown in FIG. 3(b) where the initial degradation temperatures of around 445±2° C. and 429±2° C. were found for the pristine polymer and the polymer nanocomposite, respectively.

Stress-strain curves for the dry electrode and pristine polymer samples after mechanical testing are presented in FIG. 3(c). Addition of a small percentage of CNT, which exhibits higher stiffness and strength, into the polymer matrix resulted in an increase in the elastic modulus and yield strength of the polymer composite film compared to the pristine polymer film. The high tensile and yield strength of the dry polymer nanocomposite electrode could contribute to the durability of these electrodes in wearable applications. The stiffness represented by the elastic modulus was found to be around 1.19±0.04 Gpa and 0.92±0.05 Gpa for the polymer composite film and the pristine polymer film, respectively. The strength of the samples presented by the yield strength was obtained around 16.3±0.1 Mpa and 12.7±0.1 Mpa for the polymer composite and pristine polymer, respectively. Elongation of the pristine polymer at lower stresses was relatively higher than the polymer composite due to higher stiffness of the polymer composite. Overall, the increased stiffness and strength of the dry electrode film material due to the small percentage addition of the CNT remained within reasonable ranges compared to the thermoplastic PVDF polymer allows for its application for electrical stimulation purposes with suitable flexibility and durability.

### Example 4: Electrical Characterization of Dry Electrodes

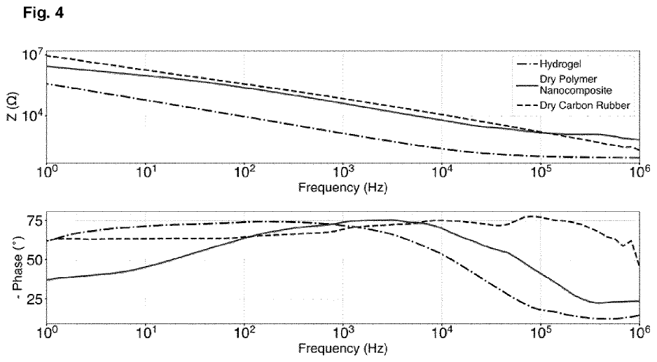

To investigate the functionality of the fabricated dry electrodes as described in Example 2, the interfacial impedance of the conductive composite material (100 μm-thick and circular with a diameter of 25 mm) was measured to ensure the desired conductivity of the layer for signal delivery to the skin. Electrical properties of three types of electrodes were determined, wherein impedance of each electrode (hydrogel, dry polymer nanocomposite as herein described, and dry carbon rubber) was measured using electrochemical impedance spectroscopy (EIS) with an electrochemical analyzer (Model CH16054E, CH Instruments, USA). The experimental setup consisted of a three-electrode configuration with one sample connected to the working electrode, a second sample connected to the counter electrode, and a third sample connected to the reference electrode while placed on a single subject's forearm (female, age 30). The parameters used were a sine wave with a peak amplitude of 0.01 V and a frequency range from 1 Hz to 1 MHz. For comparison, this procedure was performed on samples with a 5×5 cm square shape design. In addition, the average surface resistivity of the samples was measured using a digital multimeter (34401A Multimeter, Agilent, USA). From the electrical testing, the average impedance of the hydrogel electrodes for the frequency range tested was 36.06±77.70 kf, for the dry polymer nanocomposite samples the average impedance was 401.19±664.63 kf, and for the dry carbon rubber electrodes the average impedance was 970.51±1933.12 kf. The impedance and phase results are shown in FIG. 4. In addition, the average surface resistivity of the samples measured was 1.46±2.06 MΩ for the hydrogel electrode, 261.66±85.42 0 for the dry polymer nanocomposite electrode, and 628.33±198.74 kΩ for the dry carbon rubber electrode.

Based on non-limiting theory, it is hypothesized that relatively small differences among samples could be attributed to non-identical mixing processes occurring during sonication. The impedance and phase results shown in FIG. 4 indicate that the samples had desirable properties for a range of targeted applications [5]. The high impedance of the dry polymer nanocomposite can prevent high current concentrations on the skin and potentially reduce discomfort. Therefore, PVDF-CNT conductive polymeric dry electrode film can be useful as interface electrodes between a stimulator connection and skin for electrical stimulation or FES delivery.

### Example 4: Functional Electrical Stimulation (FES) Testing

To investigate functionality and efficiency for FES purposes of the fabricated dry electrodes as described in Example 2, thirteen healthy individuals were stimulated on their upper arm while measuring muscle torque and perceived comfort or discomfort levels using different electrode types and stimulator combinations. Among the subjects were five male and eight female participants whose demographics can be seen in Table 1. All participants were informed of the protocols to be used before any testing and signed an informed consent form which was approved by University Health Network's Research Ethics Board (ID #21-5298).

TABLE 1
Participant demographics.

| Age | | Weight | Height | Dominant |
|---|---|---|---|---|
| (years) | Sex | (kg) | (m) | Hand |
| 48 | Male | 96 | 1.82 | Right |
| 29 | Female | 65 | 1.65 | Right |
| 31 | Female | 50 | 1.63 | Right |
| 24 | Female | 59 | 1.70 | Right |
| 26 | Female | 63 | 1.72 | Right |
| 26 | Female | 50 | 1.50 | Right |
| 29 | Female | 50 | 1.57 | Right |
| 25 | Male | 77 | 1.80 | Right |
| 57 | Male | 95 | 1.85 | Left |
| 31 | Male | 75 | 1.73 | Right |
| 26 | Male | 75 | 1.78 | Right |
| 35 | Female | 75 | 1.60 | Right |
| 29 | Female | 51 | 1.60 | Right |

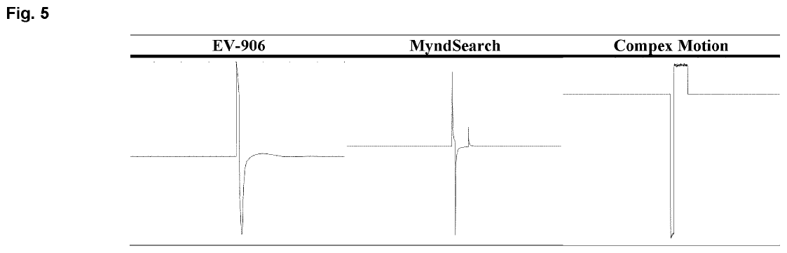

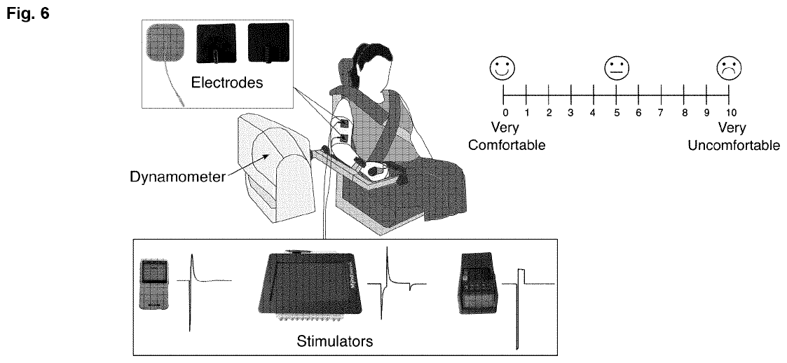

Three different electrode types were used: standard self-adhesive hydrogel (ValuTrode 5×5 cm, Axelgaard Manufacturing, Denmark), carbon rubber without electrolyte (i.e., gel or water) added (5×5 cm, AMG Medical Inc, Canada), and dry polymer nanocomposite electrodes as herein described. Each electrode type was tested with each of the three following stimulators: a portable battery-operated stimulator (EV-906, Everyway Medical Instruments, Taiwan), a MyndSearch stimulator (MyndTec Inc, Canada), and a Compex Motion stimulator (Compex SA, Switzerland). The three stimulator parameters were set at a pulse frequency of 40 Hz, pulse width of 300 μs, and pulse amplitude dependent on each participant (e.g., 3.5-28 mA). Each stimulator had a specific pulse shape as shown in FIG. 5. The EV-906 stimulator generated voltage-regulated pulses, the MyndSearch generated stimulation pulses that are both voltage and current regulated (voltage regulation is controlled by the inner loop and current regulation is controlled by the outer loop of a closed-loop controller), and the Compex Motion generated current regulated stimulation pulses.

As shown in FIG. 6, a pair of electrodes was placed on each subject's right biceps brachii, with the anode placed on the proximal end of the muscle and the cathode on the distal end of the muscle belly. After the first electrode type was tested, the outline of the electrode placement was marked to position all other electrode types in the same area. The carbon rubber electrodes and the dry polymer nanocomposite electrodes were fixated to the skin using medical tape along the sides. All electrode types, including the self-adhesive hydrogel electrodes, were wrapped with self-adherent wrap to keep them in place.

For each individual, sensory threshold (i.e., the amplitude when the person started to feel any sensation related to the stimulation), minimum contraction threshold (i.e., mCT, the amplitude when the person's muscles started to twitch or contract), and maximum tolerated contraction threshold (i.e., MTC, the amplitude when the person could not tolerate any further increase in stimulation intensity) was determined for each combination of electrode-stimulator in a randomized order.

The Biodex dynamometer (System 3, Biodex Medical Systems, USA) was then used to measure the muscle torque generated during an isometric elbow flexion. Each participant was seated in the Biodex dynamometer's chair with straps placed across their torso to hold their position in place, their arm on the chair's armrest in a supine position, and their hand holding the arm attachment's handle and a self-adhesive bandage wrapped around their hand to hold it in place. The experimental setup is shown in FIG. 6. Based on each individual's stimulation thresholds, three different intensity levels to test for each electrode-stimulator combination (low, moderate, and high) were determined based on the following formulas:

$$Low = mCT + 0.25 * ( MTC - mCT )$$

$$Moderate = mCT + 0.5 * ( MTC - mCT )$$

$$High = mCT + 0.75 * ( MTC - mCT )$$

Before stimulating at the determined intensity levels, participants performed three voluntary contractions using their maximum effort and held each for five seconds. The average of the three maximum voluntary contractions was used to normalize the stimulation-induced torque recordings for each individual. The testing order of the electrode type and stimulator was randomized for each individual. Each electrode type was tested with all three stimulators before changing the electrode type. With each combination, each subject was stimulated once to the maximum tolerated level established previously, and then three times at each intensity level (low, moderate, and high) in a randomized order. Stimulation was held for five seconds after reaching the desired intensity level. After each stimulation round, participants rated their comfort level using a visual analog scale from 0 to 10 displayed in front of them where 0 was very comfortable and 10 was very uncomfortable, shown in FIG. 6. After all rounds of stimulation with an electrode-stimulator combination, ten of the participants were asked to answer an adapted version of the short-form McGill Pain Questionnaire used for electrical stimulation and shown in Table 2 below, where there were fourteen sensation options (‘throbbing’, ‘shooting’, ‘pricking’, ‘stabbing’, ‘sharp’, ‘cramping’, ‘gnawing’, ‘pulling’, ‘hot-burning’, ‘tingling’, ‘stinging’, ‘aching’, ‘tender,’ and ‘splitting (cutting)’). Each sensation was rated on a level from 0 to 3 (0=none, 1=mild, 2=moderate, and 3=severe), depending on how much the subject felt each one.

TABLE 2
Questionnaire used to describe sensations felt during stimulation.

| | None | Mild | Moderate | Severe |
|---|---|---|---|---|
| Sensation | 0 | 1 | 2 | 3 |

Throbbing
Shooting
Pricking
Stabbing
Sharp
Cramping
Gnawing
Pulling
Hot-Burning
Tingling
Stinging
Aching
Tender
Splitting
(cutting)

Torque data was recorded from the analog outputs of the Biodex using a data acquisition system software (LabChart, PowerLab, AD Instruments, USA) at a sampling rate of 1 kHz. All data was then analyzed using MATLAB (v.2021a, Mathworks, USA). For the statistical analysis of the generated torque and comfort ratings, a Kruskal-Wallis test was used.

### Stimulation Intensities

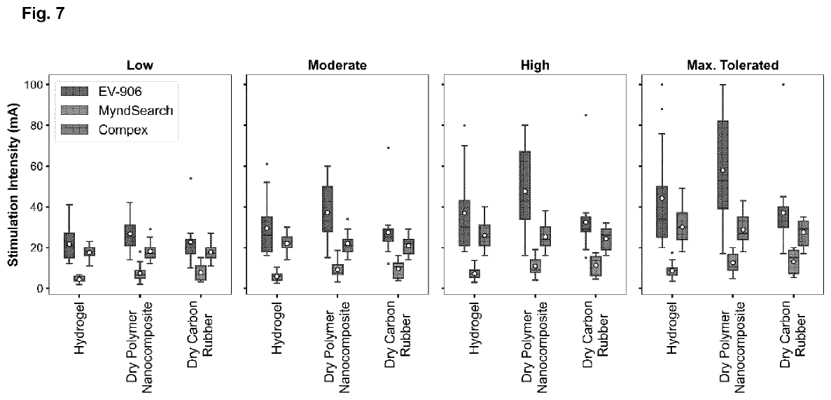

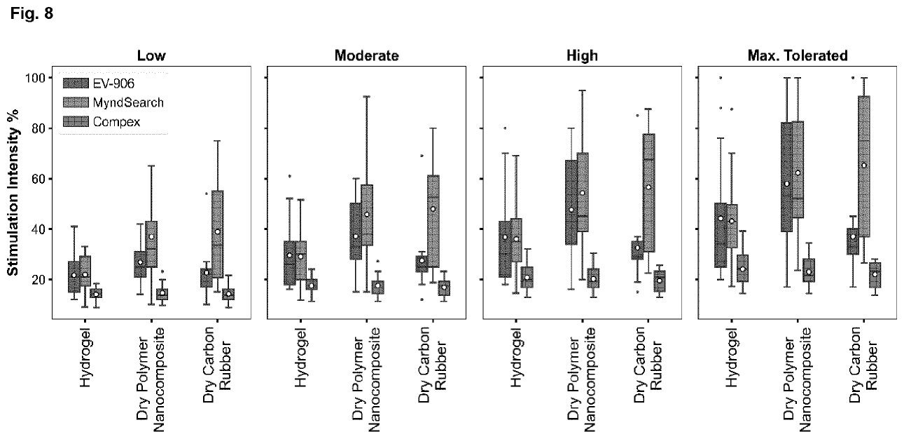

Since different stimulation intensities were used for each combination of electrode-stimulator, the average current amplitudes were compared for all individuals at each intensity level as shown in FIG. 7. Overall, higher intensities could be used with the dry polymer nanocomposite electrode with the EV-906 and the MyndSearch stimulators. For the Compex Motion, all electrode types used similar intensities on average. However, it should be noted that each stimulator had an established pulse intensity limit, and four individuals reached said limits with some electrode-stimulator combinations. One participant reached the maximum output from the MyndSearch stimulator with the dry polymer nanocomposite electrode only, one participant reached the maximum output of the EV-906 stimulator and the MyndSearch stimulator both with the dry polymer nancomposite electrode, one participant reached the maximum output of the EV-906 stimulator with the hydrogel, the dry polymer nanocomposite, and the carbon rubber electrodes, and one participant reached the maximum MyndSearch stimulator output with the carbon rubber electrodes. The EV-906 device limit was 100 mA, the MyndSearch limit was 20 mA, and the Compex Motion limit was 125 mA. If each intensity is expressed as a percentage of the maximum possible output of each stimulator, as shown in FIG. 8, the dry polymer nanocomposite electrode and the dry carbon rubber electrode allowed using higher intensities in combination with the MyndSearch given the device's limits.

### Perceived Comfort Ratings

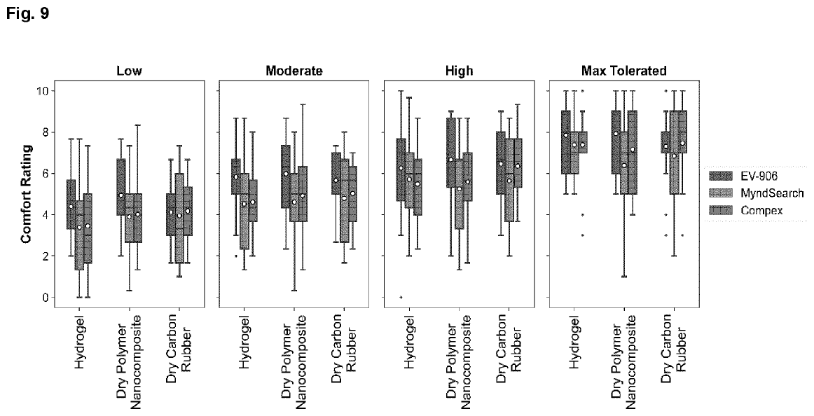

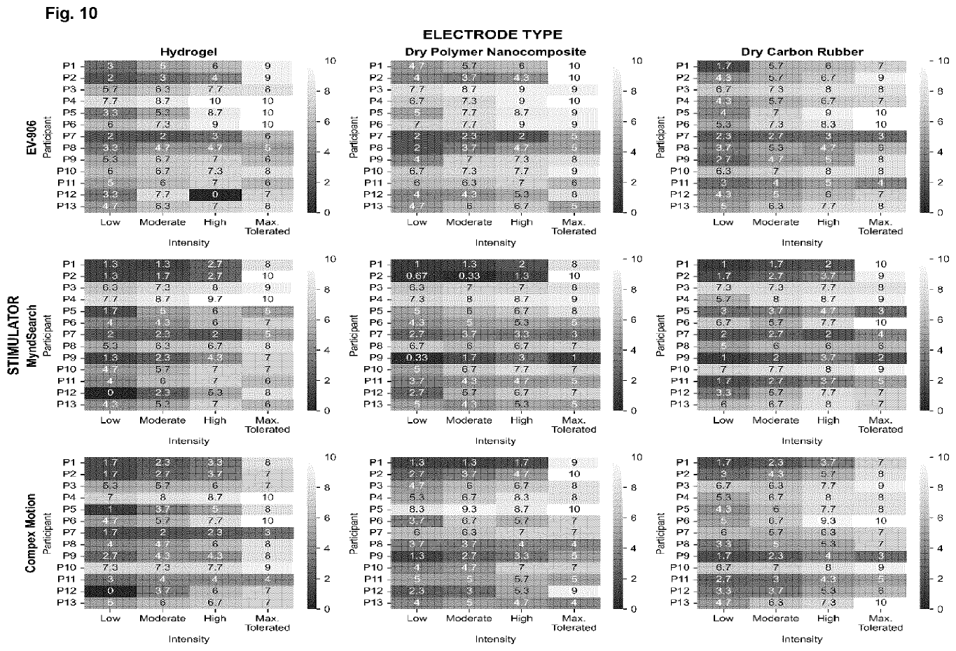

The average perceived comfort rating between all individuals for each electrode-stimulator combination is shown in FIG. 9. Based on the averages from the visual analog scale, throughout all intensity levels, the MyndSearch stimulator was the most comfortable compared to the other two stimulators. At low intensities the MyndSearch with the hydrogel electrode was the most comfortable combination with a total average comfort rating of 3.38±2.27. At moderate intensities, the MyndSearch with the hydrogel electrode was the most comfortable combination with a total average comfort rating of 4.51±2.42. At high intensities, the MyndSearch with the dry polymer nanocomposite electrode was the most comfortable combination with a total average comfort rating of 5.26±2.28. At the maximum tolerated intensity level, the most comfortable combination was the dry polymer nanocomposite electrode with the MyndSearch stimulator with a total average comfort rating of 6.38±2.50. Each individual's comfort rating for each combination can be found in FIG. 10. After doing a statistical analysis, no statistically significant difference (p<0.05) was found in the comfort ratings between the three electrode types used, or the three different stimulators used. The p-values for each electrode or stimulator tested can be found in Table 3.

TABLE 3
Statistical analysis p-values for comfort ratings.

| | | Low | Moderate | High | Maximum |
|---|---|---|---|---|---|
| | Electrode | Inten- | Inten- | Inten- | Tolerated |
| | Stimulator | sity | sity | sity | Intensity |
| Difference | Hydrogel | p = 0.278 | p = 0.156 | p = 0.449 | p = 0.766 |
| between 3 | Dry polymer | p = 0.378 | p = 0.234 | p = 0.187 | p = 0.210 |
| stimu- | nanocom- | | | | |
| lators | posite | | | | |
| used | Dry carbon | p = 0.951 | p = 0.639 | p = 0.636 | p = 0.903 |
| | Rubber | | | | |
| Difference | EV-906 | p = 0.466 | p = 0.734 | p = 0.912 | p = 0.671 |
| between 3 | MyndSearch | p = 0.788 | p = 0.872 | p = 0.778 | p = 0.677 |
| electrodes | Compex | p = 0.637 | p = 0.751 | p = 0.501 | p = 0.938 |
| types used | Motion | | | | |

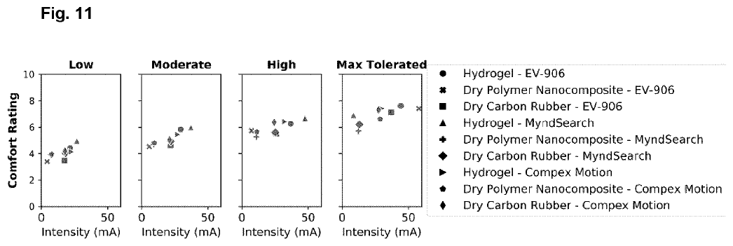

A comparison between the intensity of stimulation applied and the comfort level can also be seen in FIG. 11. The EV-906 stimulator had the biggest differences between intensities by electrode type used, but the comfort level was very similar between them. The MyndSearch stimulator required higher intensities with the dry electrodes than with the hydrogel, but both the dry polymer nanocomposite electrode and the dry carbon rubber electrode were more comfortable at the maximum tolerated intensity level. The Compex Motion stimulator used similar intensities for all electrode types, but the carbon rubber electrode was the most uncomfortable at all levels, and the dry polymer nanocomposite was more comfortable at the maximum tolerated level.

Most transcutaneous electrode types require the addition of gel or water to improve the interface with skin and result in less discomfort. However, use of a completely dry polymer nanocomposite electrode as herein disclosed can perform as well as the standard self-adhesive hydrogel electrodes for stimulation purposes.

### Stimulation-Induced Muscle Torque

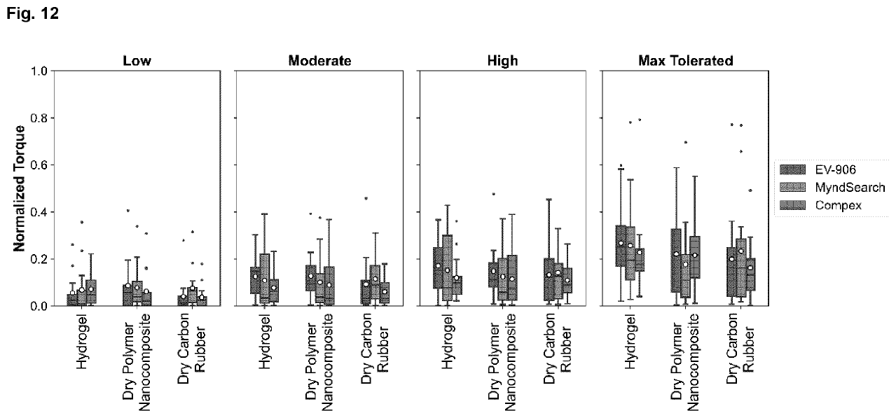

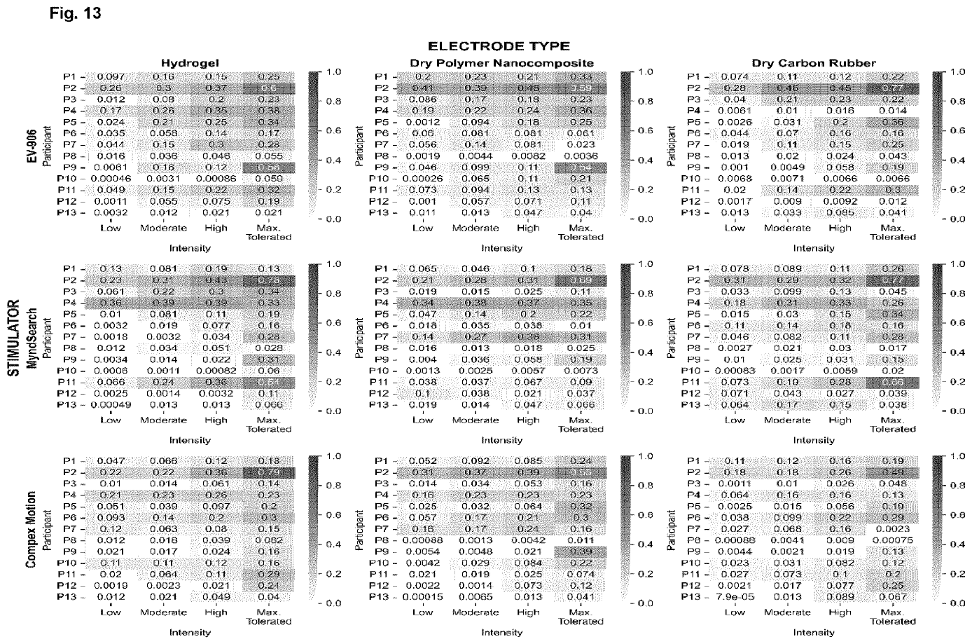

From the stimulation-induced torque values recorded, the average steady-state torque of each individual was used to do the comparison. The steady-state torque was defined as the average torque within the last second of stimulation at the intensity level being tested. The average of the three maximum voluntary contractions was used to normalize the torque for each individual. The stimulation-induced normalized torques are presented in FIG. 12. At the low intensity level, the dry polymer nanocomposite electrode with the EV-906 stimulator generated the strongest contractions on average at 0.086±0.116. At moderate intensity, the dry polymer nanocomposite electrode with the EV-906 stimulator generated the strongest contractions on average at 0.127±0.105. At high intensity, the hydrogel electrode with the EV-906 stimulator generated the strongest contractions at 0.171±0.121. At the maximum tolerable intensity, the hydrogel electrode with the EV-906 stimulator generated the strongest contractions at 0.267±0.181. Each individual participant's generated torque for each combination can be seen in FIG. 13. After doing a statistical analysis to see if there was a significant difference in generated torque depending on the electrode type or stimulator used, no statistically significant difference (p<0.05) was found as shown on the p-values on Table 4.

TABLE 4
Statistical analysis p-values for normalized generated torque.

| | | Low | Moderate | High | Maximum |
|---|---|---|---|---|---|
| | Electrode | Inten- | Inten- | Inten- | Tolerated |
| | Stimulator | sity | sity | sity | Intensity |
| Difference | Hydrogel | p = 0.419 | p = 0.491 | p = 0.634 | p = 0.711 |
| between 3 | Dry polymer | p = 0.699 | p = 0.258 | p = 0.414 | p = 0.526 |
| stimu- | nanocom- | | | | |
| lators | posite | | | | |
| used | Dry carbon | p = 0.141 | p = 0.309 | p = 0.825 | p = 0.816 |
| | Rubber | | | | |
| Difference | EV-906 | p = 0.649 | p = 0.329 | p = 0.673 | p = 0.370 |
| between 3 | MyndSearch | p = 0.613 | p = 0.344 | p = 0.550 | p = 0.813 |
| electrodes | Compex | p = 0.381 | p = 0.806 | p = 0.848 | p = 0.423 |
| types used | Motion | | | | |

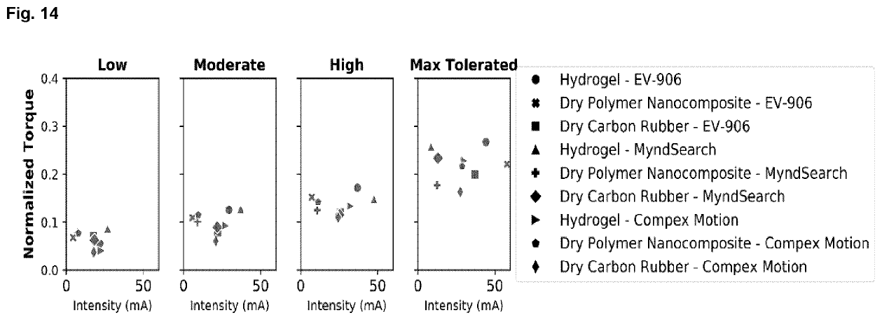

Comparing the intensity of stimulation applied to the average amount of torque generated, shown in FIG. 14, the hydrogel electrodes produced the highest amount of torque with lower intensities than the dry electrode types at the high and maximum tolerated levels for the EV-906 and MyndSearch stimulators. While for the Compex Motion stimulator, slightly higher intensities could be used with the hydrogel electrodes, but the amount of torque was also higher.

Although the differences were not statistically significant, the combination that produced the highest torque and was rated as the most comfortable at the maximum tolerated stimulation level was the dry polymer nanocomposite electrode with the MyndSearch stimulator.

### Reported Sensations

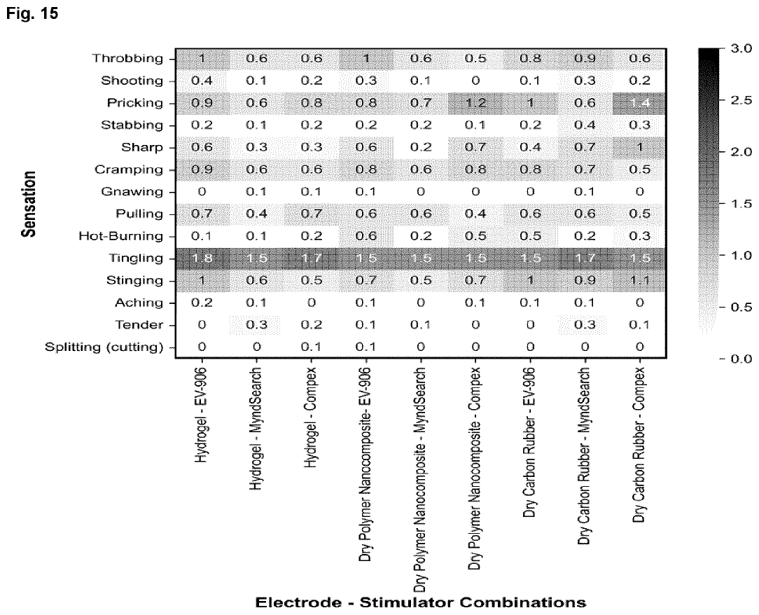

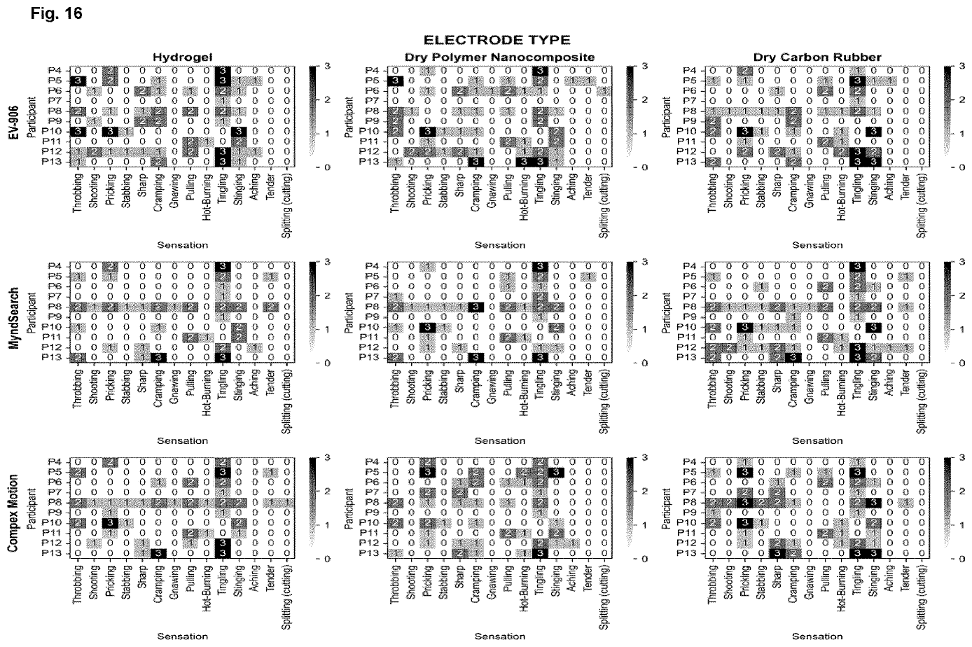

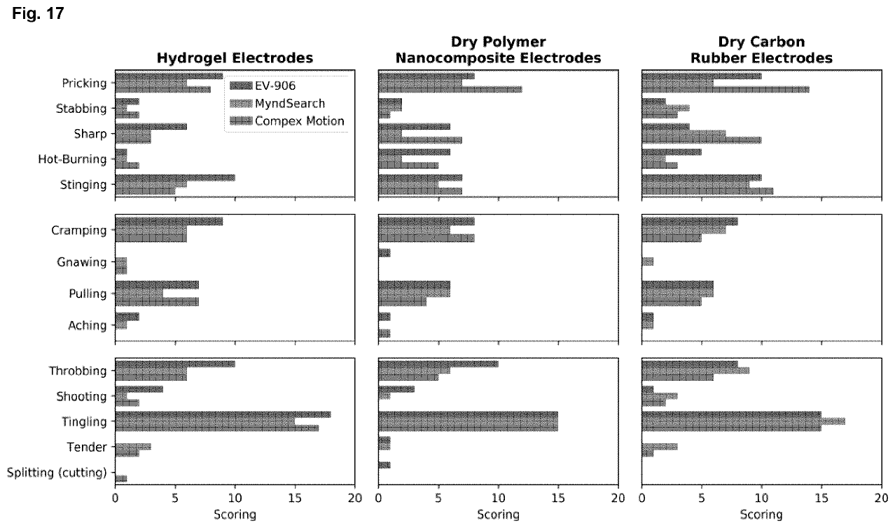

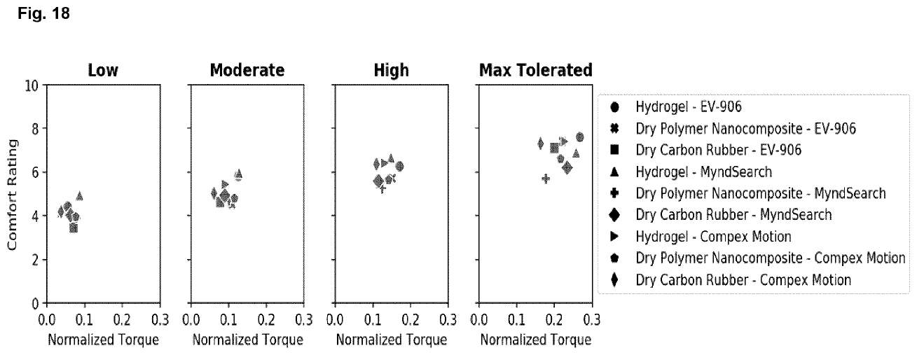

The average reported sensations from the given questionnaire are shown in FIG. 15. ‘Tingling’ was the most frequently reported sensation for all possible combinations and had very similar scores between 1.5-1.8 out of 3. Each individual participant's reported sensations can also be found in FIG. 16.

The described sensations can be separated into three different categories as they are related to the level of nerve fibers activated: cutaneous (superficial), muscular (deep), and general. Cutaneous sensations include ‘pricking’, ‘stabbing’, ‘sharp’, ‘hot-burning’, and ‘stinging’. Particularly, ‘pricking’ and ‘stinging’ have been associated with activation of Aδ fibers while ‘stabbing’, ‘sharp’, and ‘hot-burning’ have been associated with C fibers. Muscular sensations include ‘cramping’, ‘gnawing’, ‘pulling’, and ‘aching’. These are all related to the activation of deep nociceptors and muscle pain. General sensations are ‘throbbing’, ‘shooting’, ‘tingling’, ‘tender,’ and ‘splitting (cutting)’, which are all associated with the stimulation of mechanoreceptors. A combined added score of all sensations per category can be found in FIG. 17. All electrode types with all stimulators reported more cutaneous than deep sensations. In particular, the dry carbon rubber electrode with the Compex Motion had the most amounts of reported ‘pricking’, ‘sharp’ and ‘stinging’ sensations. Within the deep category, ‘cramping’ and ‘pulling’ were the most frequently reported sensations with all electrode and stimulator types.

FIG. 18 shows average amount of normalized torque generated in relation to its perceived comfort level.

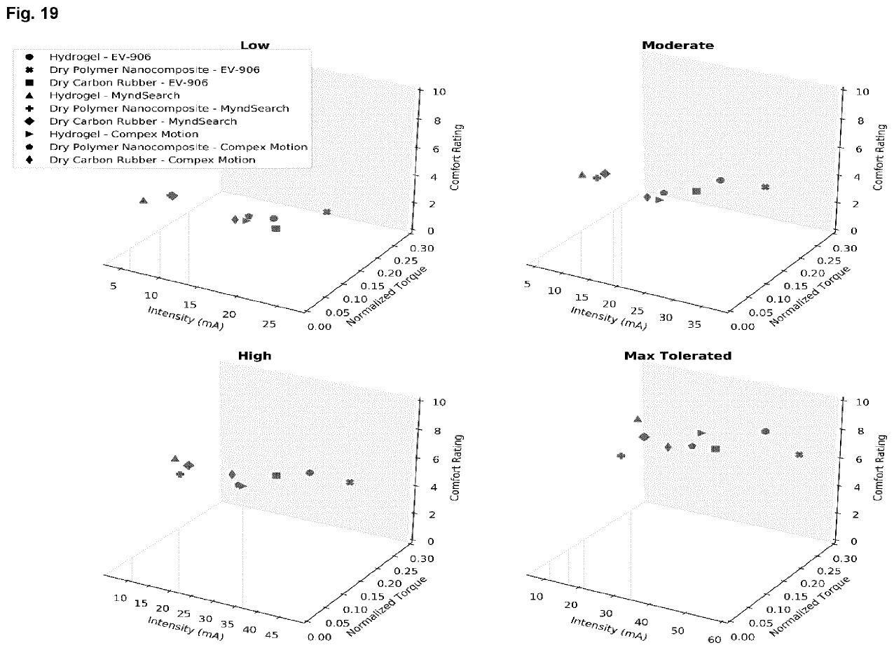

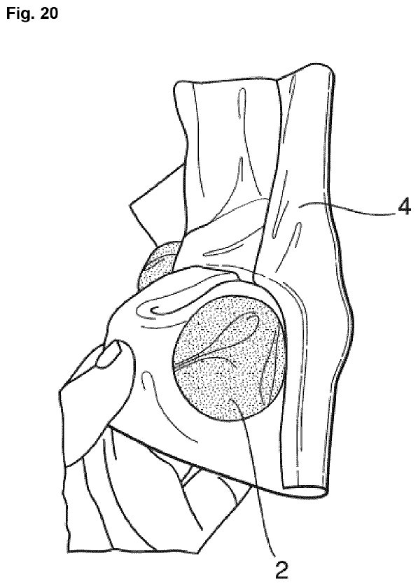

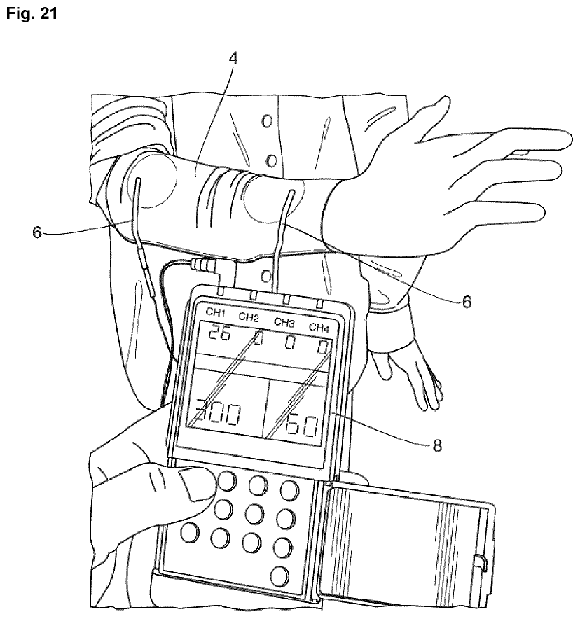

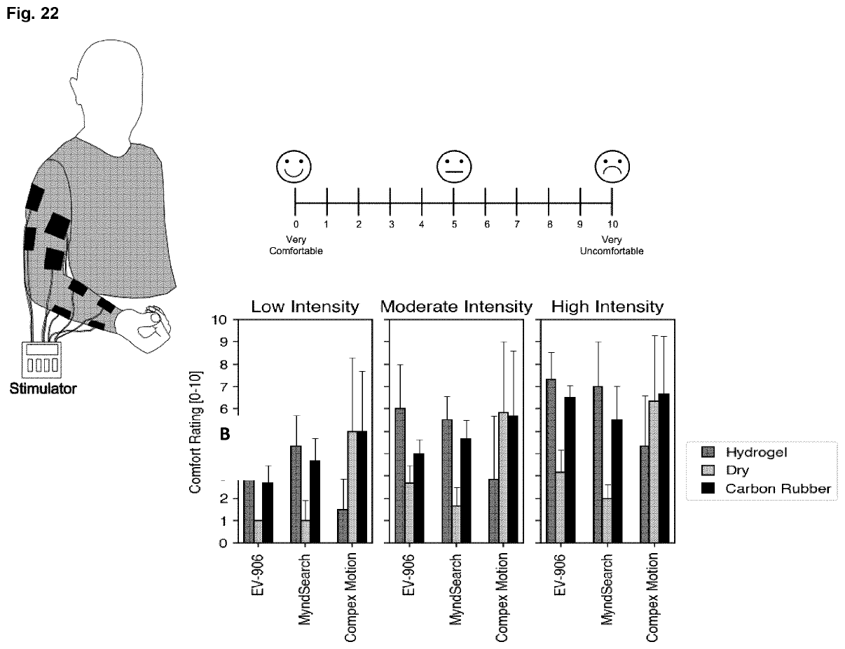

FIG. 19 shows 3D-plots showing average amount of torque generated vs. comfort rating vs. intensity of stimulation used at each intensity level.

Overall, the dry polymer nanocomposite electrode had a high yield strength and sufficient flexibility, while also having a high impedance. It had a smooth surface and did not require the addition of gel or water to work as well as the current self-adhesive hydrogel electrodes available.

### Example 5: Manufacture of Dry Electrode Articles or Garments

The herein-described dry polymer nanocomposite electrode can be integrated into garments for stimulation, for example due to its reusable nature and smooth non-adhesive surface, and can potentially enable a more user-friendly form to deliver FES. When integrated into a garment, it could also facilitate the use of stimulation in settings outside the clinic.

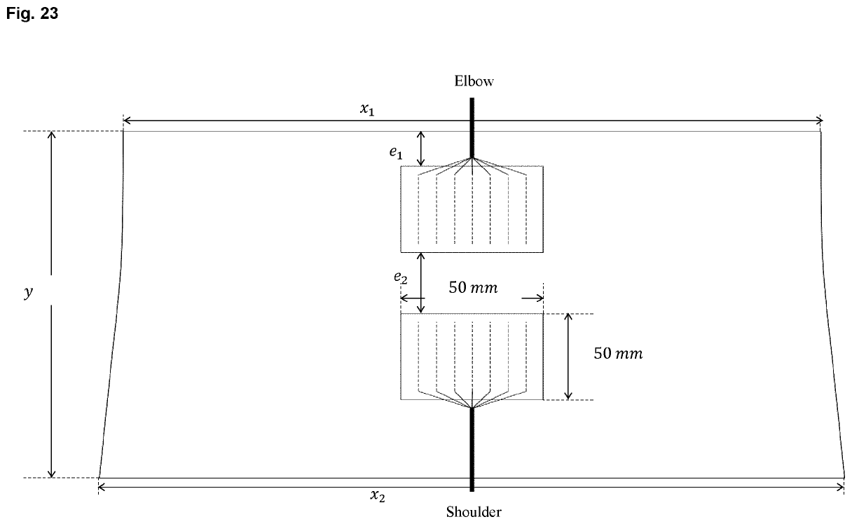

FIG. 20 shows attachment of a dry electrode onto the inner side of a stretchable fabric garment to interface with the skin. In this configuration, the sleeve was designed to apply electrical stimulation through a forearm. The position of the dry electrode was selected based on the anatomy of a target muscle. The dry electrode attachment was performed either by applying commercial adhesives between the garment and the back of the electrode or by heating the electrode placed on the garment to 180 to 200° C. for 4 to 8 minutes and simultaneously compressing the film layer onto the garment under a force of 2 to 6 tons using a compression molding setup. Prior to attachment, one end of the conductor was run and held between the garment and back of the dry electrode to be fixed to the electrode through the attachment to the garment. The other end of the conductor was connected to a stimulator.

FIG. 21 shows the outer side of the garment 4 shown in FIG. 20 wherein the sleeve was worn by a participant in such a way that the dry electrodes were in direct contact with the desired locations on the arm of the wearer and the conductor ends were connected to a stimulator for applying electrical pulses. The stretchable garment allowed for adjustment and proper positioning of the electrodes in close proximity to the target muscles.

### Example 6: Use of Dry Electrode Device

The dry electrodes described in Example 5 were arranged on a participant such to come into close proximity with muscles of interest responsible for performing a particular motion, for example, a reaching-to-the-mouth motion. To perform a left-hand reaching movement toward the mouth, pairs of electrodes were placed on the front deltoid muscle, biceps and triceps of the left arm. By simultaneously stimulating the front deltoid muscle and biceps, the arm flexed about the elbow (contraction of the biceps muscle), and the arm gently twisted medially so that the arm could reach the mouth (contraction of the front deltoid muscle). To move the arm away from the mouth and extend it next to the body, stimulation was stopped in the front deltoid and biceps muscles to produce elbow extension by stimulating the triceps muscle. The electrodes responsible for stimulating these three muscles were all embedded into an elastic garment that resembled a sleeve covering all of the foregoing muscles. Those skilled in the art of functional electrical stimulation (FES) are skilled in placing electrodes to generate movements such as, by way of example, hand opening and closing, reaching, standing up, walking, and sitting. An elastic dry electrode garment covering the areas of the body containing the target muscles such as that shown in FIGS. 20 and 21 facilitates desirable electrode placement required to stimulate target muscles by placement above the targeted muscles.

### Example 7: Functional Electrical Stimulation (FES) Testing

To determine functionality of the dry electrodes comprising conductive composite material (as described in Example 3) during FES, stimulation on the upper limb of two healthy participants (one male and one female) was tested with a mean age of 38.5±13.4 years. Comfort levels were compared using standard self-adhesive hydrogel electrodes (ValuTrode 5×5 cm, Axelgaard Manufacturing, Denmark), dry commercial carbon rubber electrodes (5×5 cm, AMG Medical, Canada), and the dry electrodes of interest. As illustrated in FIG. 6, participants were seated on a Biodex chair with the backrest straight with a hand holding the arm attachment's handle in a supine position. A self-adhesive bandage was wrapped around the hand to hold it in place. The armrest height was adjusted for each participant so that their shoulder would be in a comfortable neutral straight position. The dynamometer's axis of rotation was aligned to each individual's elbow. Two straps were placed across each participant's torso to maintain their position.

To determine stimulation sensory thresholds of participants, a pair of electrodes was placed on the right biceps brachii of each participant. The anode was placed on the proximal end of the muscle (i.e., on top of the long and short heads of the brachii), while the cathode was placed on the distal end of the muscle belly. Dry electrodes as herein described were placed directly against the participant's skin. The outline of electrode placement was marked on the skin to ensure that all electrode types tested were placed in the same position. The electrodes were wrapped with self-adhesive bandages to secure them in place when placed on the arm.

Three different stimulators with asymmetric biphasic pulses were used: a portable battery-operated stimulator (EV-906, Everyway Medical Instruments, Taiwan-voltage regulated stimulator), a MyndSearch stimulator (MyndTec, Canada—voltage and current regulated stimulator), and a Compex Motion stimulator (Compex, Switzerland—current regulated stimulator). Examples of pulses delivered using these stimulators are shown in FIG. 5. The stimulators were used such that they represent all regulation modalities for electric stimulators. Stimulator parameters were set at a pulse frequency of 40 Hz, pulse width of 300 μs, and pulse amplitude dependent on each participant and stimulator used (3.5-28 mA).

Individual sensory thresholds were determined (i.e., the amplitude when the participant started feeling the stimulation), minimum contraction thresholds (i.e., mCT, amplitude when the selected muscle first started to twitch or contract), and maximum tolerated contraction thresholds (i.e., MTC, amplitude when the stimulation was too uncomfortable for the participant). These thresholds were determined with the standard hydrogel electrodes in combination with each of the three stimulators in order to test the same intensity levels with all three electrode types. Based on these thresholds, three different intensities (low, moderate, and high) were calculated to stimulate each participant with:

$$Low : mCT + 0.25 * ( MTC - mCT )$$

$$Moderate : mCT + 0.5 * ( MTC - mCT )$$

$$High : mCT + 0.75 * ( MTC - mCT )$$

The type of electrode, stimulator, and intensity order were randomized. Participants performed three initial voluntary contractions using their maximum effort and held each for five seconds. Then the participant was stimulated with one type of electrode at each intensity three times. The stimulation amplitude was increased 0.25-1 mA at a time until the desired intensity was reached and then held for five seconds. After each round of stimulation, participants would report verbally how comfortable the stimulation felt using a visual numerical rating scale from 0 to 10 displayed in front of them, where 0 would be very comfortable and 10 very uncomfortable. Each electrode type was tested with the three different stimulators, and once one type of electrode was tested for all intensities, the electrode type was changed and the same stimulation procedure described before was repeated.

A data acquisition system and software (PowerLab, LabChart, AD Instruments, USA) was used to record the data. Torque was recorded at 1 kHz sampling rate, while stimulation pulses were recorded at 200 kHz. All data was collected and stored for further analysis in a computer.

After data collection, MATLAB (v.2021a, Mathworks, USA) was used to filter and analyze the data.

### Comfort Ratings

As shown in FIG. 22, across all intensity levels, the dry electrodes were perceived as the most comfortable with the MyndSearch and the EV-906 stimulators, while the hydrogel electrode was the most comfortable with the Compex Motion stimulator. The most uncomfortable combinations were the dry electrodes and dry carbon rubber electrodes with the Compex Motion stimulator.

At the end of all the stimulation tests, both participants reported the combination of the MyndSearch and the dry electrode as the most comfortable of all, which is consistent with the ratings shown in FIG. 22.

### Example 8: Functional Electrical Stimulation Bicep Sleeve Based on Textile-Embedded Dry Electrodes

This example presents a design and prototyping method for a smart bicep sleeve for FES applications and explains how to integrate a carbon-based dry electrode into a textile structure and ensure an electrical connection between the electrodes and the stimulator for effective delivery of the FES.

### Materials and Methods

### Dry Electrode

The dry electrode used to develop the smart FES garment was made as described above. Briefly, it was made of Polyvinylidene Fluoride (PVDF) mixed with 5% in weight of Carbon nanotube (CNT). Then, the polymer was shaped into a thin, flexible, and conductive film which can be easily tailored to desired sizes.

### Textile Conductive Yarn

While sewing or embroidering conductive textile yarns has been widely used to create conductive tracks on textile substrates [30-32], prior art methods have not employed a sewn conductive matrix as a contact power supply support for dry electrodes as for the herein-described FES garment. As shown in FIG. 1, the conductive matrix of the smart FES garment consisted of 7 parallel stitch lines made with two “Silver-Tech HC12” yarns (sewing and bobbin), from Madeira Company [33]. This yarn presents a linear resistivity around 100 Ω·m-1 [34]. Use of conductive threads for both sewing and bobbin allows for an electrical connection between both sides of the42easuree, thus enabling connection of both the electrode inside the sleeve and stimulation outside the sleeve.

### Textile Substrate

A blend of Polyester (80%), Viscose (15%) and Elastane (5%) was used to develop the smart FES sleeve. As a support, the textile sleeve must ensure accurate positioning and good adhesion of the electrodes, and must withstand the electrode fixation process, which includes thermal compression molding at 190° C. This structure was chosen because it provides elasticity and ensures a good skin-electrode interface.

### Textile Sleeve Pattern

The knitted substrate was cut according to a specific pattern to shape a sleeve. The textile sleeve pattern is the 2-dimensional representation of the sleeve before it was assembled, as presented in FIG. 23. It is created based on the patient's biceps measurement and the positions of the electrodes, with the aim of stimulating the anterior brachial biceps, responsible for elbow flexion. Three different sizes (S, M, L) were developed, and their dimensions are displayed in Table 1. Inkscape® software was used to design the pattern.

TABLE 5
The corresponding size guide of the smart FES biceps sleeve.

| Size | x₁ | x₂ | y | e₁ | e₂ |
|---|---|---|---|---|---|
| S | 21 cm | 23 cm | 19 cm | 2 cm | 3.5 cm |
| M | 23.5 cm | 25 cm | 20.5 cm | 2.3 cm | 4.2 cm |
| L | 26 cm | 29 cm | 21.5 cm | 2.5 cm | 5 cm |

### Textile Electrode Conductive Interface

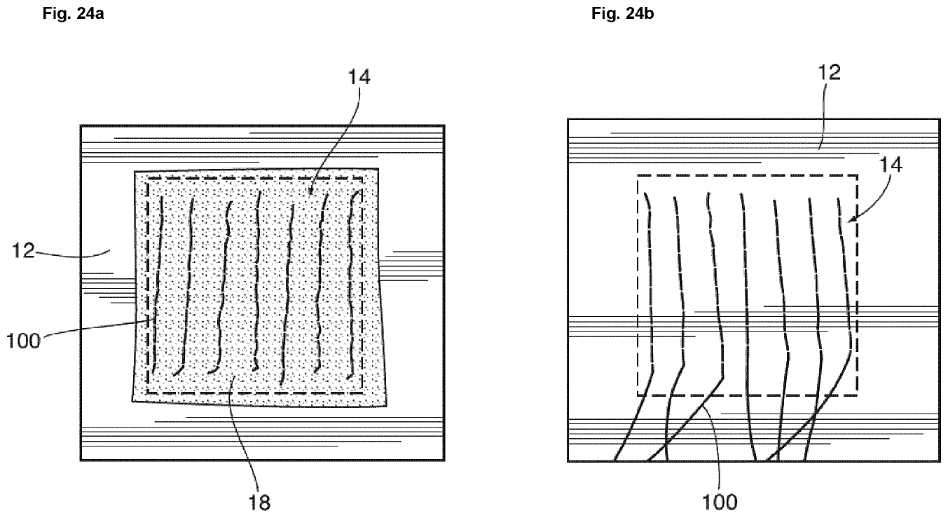

The textile conductive yarns created the electrical connection between the dry electrode and the stimulator. They formed a conductive matrix, fixed by sewing or embroidery, on the knitted substrate. FIG. 24 presents a sample of the embroidered conductive matrix on the knitted substrate, both the front and the back sides. The textile conductive yarns composing the matrix were extended to a crimp connector, allowing them to carry the current from the stimulator to the electrode. Additionally, the use of sewn textile conductive yarns provides a larger contact surface, improving the mechanical and chemical fixation. Moreover, the matrix's roughness enabled the electrode's polymer to fill the new volume under thermal compression, enhancing mechanical fixation.

The matrix was composed of seven parallel textile conductive yarns. This geometry ensured a homogeneous current distribution inside the matrix, and consequently in the electrode.

### Dry Electrode Fixinp

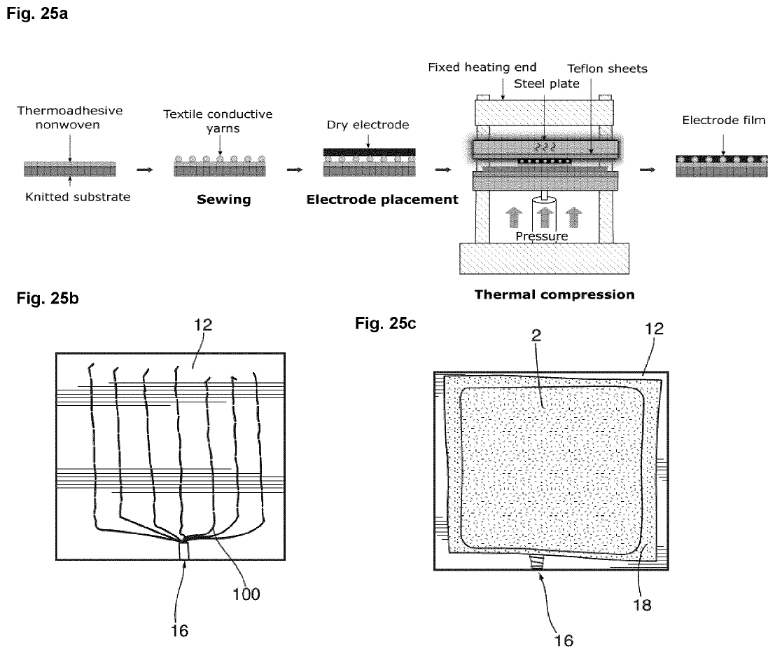

Once the textile conductive matrix was formed, the carbon-based dry electrode was fixed on the substrate through a thermal compression process, as described in FIG. 25(a), using a CARVER Compression Molding Press. The machine applied a temperature of 190° C. solely from the top side. From the bottom side, 1 ton pressure was applied for 5 minutes, followed by 5 tons pressure for 5 to 7 minutes. The area of the compression mold press was a 15×15 cm square shape. To increase the surface smoothness, two Teflon sheets were placed on the steel plate to sandwich the textile substrate and the electrode. After the thermal compression, the sample was removed once it cooled down to room temperature. Photographs of both sides of the fixed dry electrode on the knitted substrate are presented in FIGS. 25(b) and 25(c).

### Tailoring

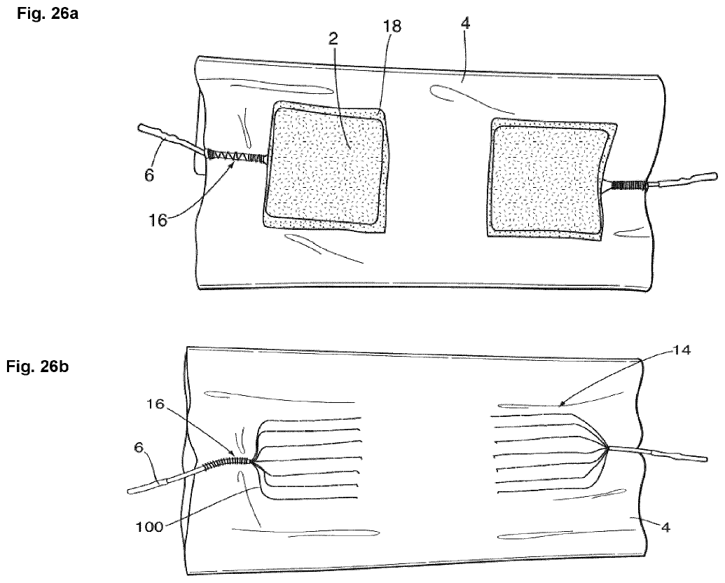

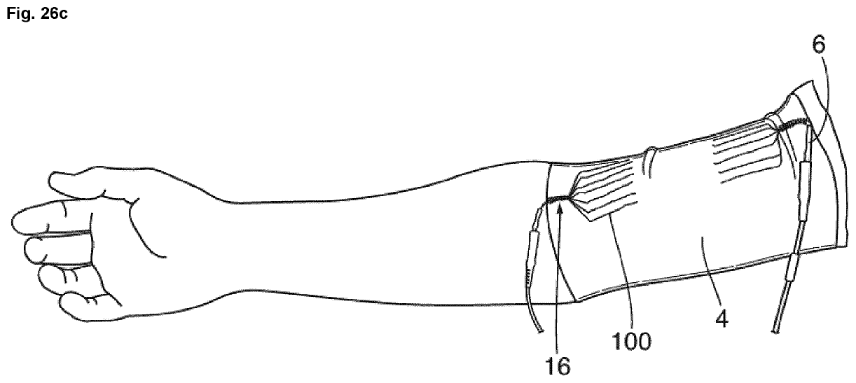

The last step of the manufacturing process consisted of tailoring the pattern to shape it into a sleeve and integrate the connectors. The remaining protruding textile conductive yarns from the conductive matrix were twisted and then inserted into a circular crimp connector, which was then threaded into shrink tubing. The resulting cables were fixed on the substrate using cording stitches. The textile structure (flat) was finally closed to form a sleeve with straight stitches. The prototype of the smart FES sleeve for biceps is presented in FIG. 26.

### Experimental Protocol of the Smart FES Sleeve Characterization

The purpose of the smart FES sleeve for the biceps was to provide an elbow flexion through electrical stimulation of the anterior biceps brachii without causing pain. The stimulation was delivered to the right arm using the MyndSearch stimulator (MyndTec Inc.), with a pulse frequency of 40 Hz, a pulse width of 300 μs, and a pulse amplitude determined for each individual. The smart FES sleeve, which embeds dry electrodes, was compared against conventional self-adhesive hydrogel electrodes (ValuTrode 5×5 cm, Axelgaard Manufacturing Co.) in terms of muscle torque and perceived comfort. The electrical intensity, the perceived comfort and the muscle torque were evaluated for 3 levels of stimulation intensity, with both the hydrogel electrodes and the smart FES sleeve for five participants, following the protocol outlined in Example 4 above. Torque was measured using a Biodex dynamometer (System 3, Biodex Medical Systems) and comfort was rated using a 1 to 10 visual analog scale displayed in front of participants as well as a sensations questionnaire. All participants were informed of the protocols and signed a consent form approved by University Health Network's Research Ethics Board (ID #21-5298). The three levels of stimulation intensity used to perform the measurements were defined as follows:

$$Low = mCT + 0.25 * ( MTC - mCT )$$

$$Moderate = mCT + 0.5 * ( MTC - mCT )$$

$$High = mCT + 0.75 * ( MTC - mCT )$$

Where mCT is the minimum contraction threshold, and MTC the Maximum Tolerated Contraction threshold.

### Participants Demographics

The smart FES sleeve was tested on five participants (3 female and 2 male, mean age 28.6±2.7 years old), as presented in Table 2.

TABLE 6
Participant demographics.

| Age | | Weight | Height | Dominant |
|---|---|---|---|---|
| (years) | Sex | (kg) | (m) | Hand |
| 32 | Male | 80 | 1.73 | Right |
| 30 | Female | 67 | 1.65 | Right |
| 25 | Female | 65.8 | 1.70 | Right |
| 29 | Female | 66 | 1.63 | Right |
| 27 | Male | 72.6 | 1.78 | Right |

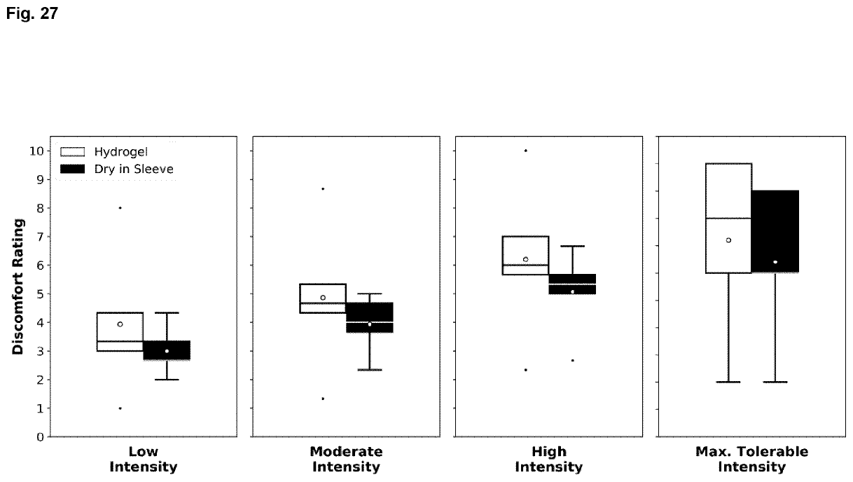

For reported comfort ratings, participants rated the sleeve with the dry electrodes to be at least as comfortable as the hydrogel electrodes on average for all intensity levels, as seen in FIG. 27. For the maximum tolerable intensity, the average discomfort was 6.40±2.88 with the sleeve, and 7.20±3.35 with the hydrogel electrodes. At high intensity, the average discomfort rating was 5.07±1.48 with the sleeve and 6.20±2.75 with the hydrogel electrodes. At moderate intensity, the average discomfort was 3.93±1.04 with the sleeve and 4.87±2.62 with the hydrogel electrodes. At low intensity, the average discomfort rating was 3.00±0.88 with the sleeve, and 3.93±2.58 with the hydrogel electrodes.

### Stimulation Induced Muscle Torque

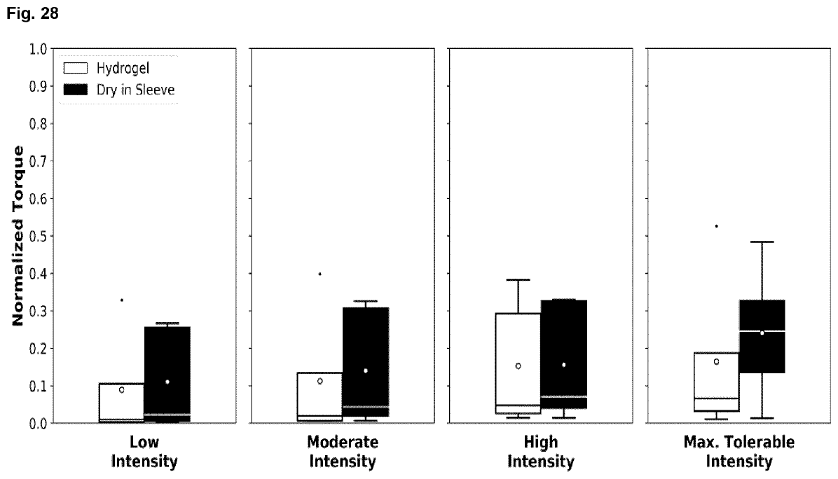

The muscle torques produced with stimulation on average were not significantly different with either electrode type at any intensity level and are shown in FIG. 28. At the maximum tolerable stimulation intensity, the average normalized torque produced was 0.241±0.180 with the sleeve and 0.164±0.213 with hydrogel electrodes. At high intensity, the average torque with the sleeve was 0.156±0.158 and 0.153±0.172 with the hydrogel electrodes. At moderate intensity, the average torque was 0.140±0.161 with the sleeve and 0.113±0.169 with the hydrogel electrodes. At low intensity, the average torque with the sleeve as 0.111±0.138 and 0.089±0.141 with the hydrogel electrodes.

### Stimulation Intensities Used

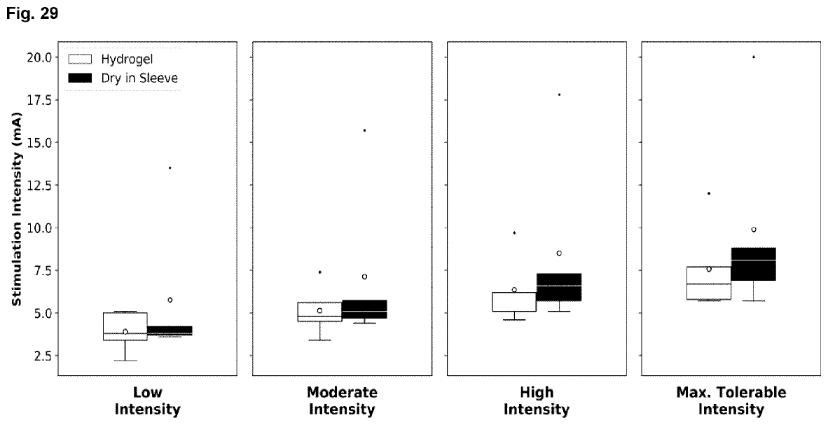

The stimulation intensities used for all five participants, seen in FIG. 29, were higher on average with the dry electrodes in the sleeve than with the hydrogel electrodes. At the maximum tolerable level, the average stimulation used was 9.90±5.77 mA with the sleeve, and 7.58±2.60 mA with the hydrogel electrodes. For the high level, the average intensity was 8.50±5.27 mA with the sleeve and 6.36±1.99 mA with the hydrogel electrodes. For moderate levels, the average intensity was 7.13±4.82 mA with the sleeve and 5.14±1.49 mA with the hydrogel electrodes. For low levels, the average intensity was 5.76±4.33 mA with the sleeve and 3.90±1.20 with the hydrogel electrodes.

### Stimulation Sensations Reported

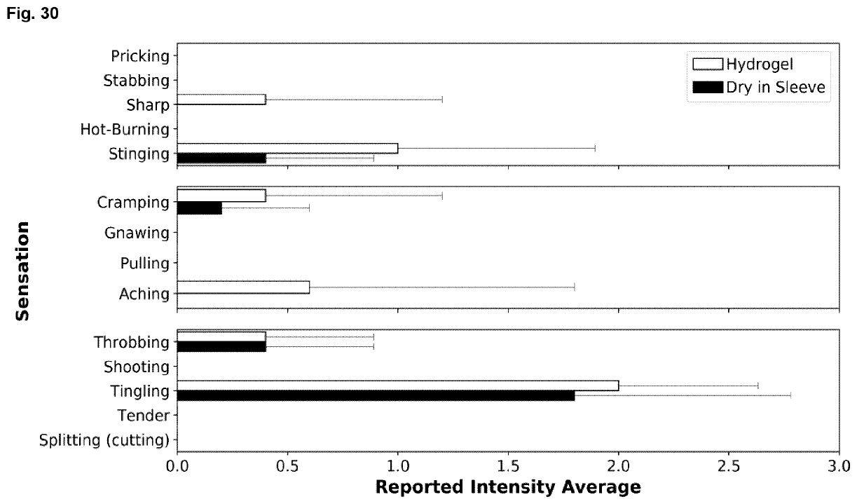

The average reported sensations after stimulation are shown in FIG. 30. The most reported sensation on average was ‘tingling’ for both electrode types, with individuals rating the intensity on average 1.80±1.10 for the sleeve and 2.00±0.71 for the hydrogel electrodes. Other reported sensations were ‘stinging’, ‘throbbing’, and ‘cramping’ with the sleeve and ‘stinging’, ‘aching’, ‘sharp’, and ‘cramping’, and ‘throbbing’ with the hydrogel electrodes. The sensation intensities were rated on average at 0.40±0.55 with the sleeve and 1.00±1.00 with the hydrogel electrodes for ‘stinging’, 0.40±0.55 with both sleeve and hydrogel electrodes for ‘throbbing’, 0.20±0.45 with the sleeve and 0.40±0.89 with the hydrogel electrodes for ‘cramping’, and 0.60±1.34 for ‘aching’ and 0.40±0.89 for ‘sharp’ with the hydrogel electrodes.

### Example 9: Functional Electrical Stimulation Forearm Sleeve Based on Textile-Embedded Dry Electrodes

This example presents a design and prototyping method for a smart forearm sleeve for FES applications.

### Materials and Methods

A dry electrode, textile conductive yarn and textile substrate were made as described in Example 8.

### Textile Sleeve Pattern

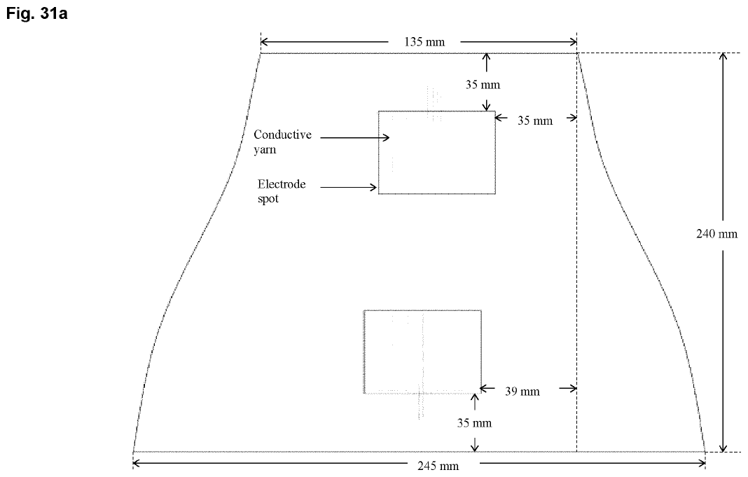

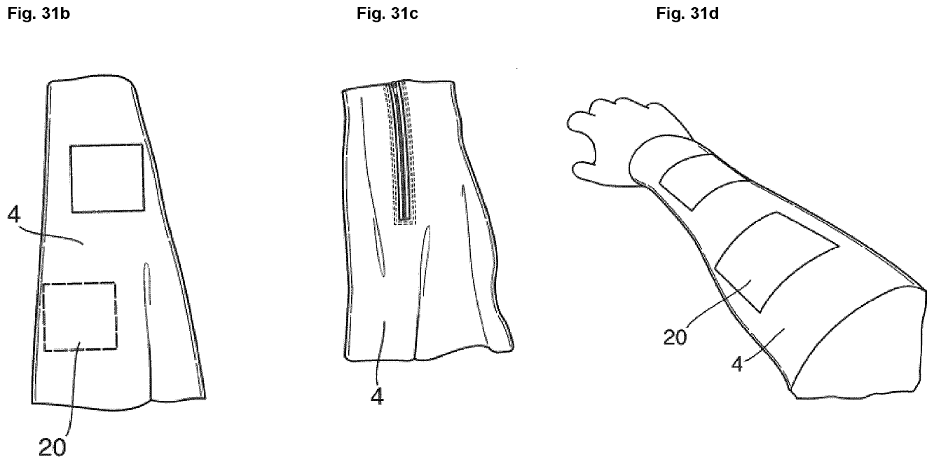

Once the electrodes have been fixed on the substrate, the knitted PET jersey was cut according to a specific pattern to shape a sleeve. The textile sleeve pattern was the 2-dimensional representation of the sleeve before it was assembled, as presented in FIG. 31(a). It was created based on the forearm measurements of the patient and the electrode positions. A sewing process took place at the end to assemble the pattern, as shown in FIGS. 31(b-d). The Inkscape™ software was used to design the pattern.

Slipping the sleeve on the forearm could be difficult because of the diameter difference between the distal (wrist) and proximal parts of the forearm, especially for patients with motor dysfunction. Therefore, the sewing on the lower forearm could be replaced by a zipper, as shown in FIG. 31(c).

### Textile Electrode Conductive Interface

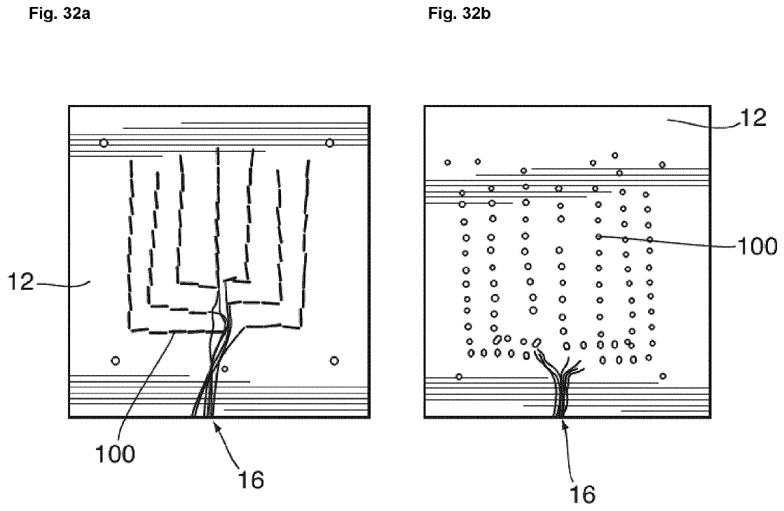

The electrical connection between the dry electrode and the stimulator was created by the textile conductive yarns. They form a conductive matrix, fixed by embroidery, on the knitted substrate. FIG. 32 presents a sample of the embroidered conductive matrix on the knitted substrate, both the front and back sides. The textile conducive yarns composing the matrix were extended to a crimp connector, allowing them to carry the current from the stimulator to the electrode. Additionally, the use of embroidered textile conductive yarn provided a larger contact surface, improving the chemical fixation. Moreover, the matrix's roughness enabled the electrode's polymer to fill this new volume under thermal compression, enhancing mechanical fixation.

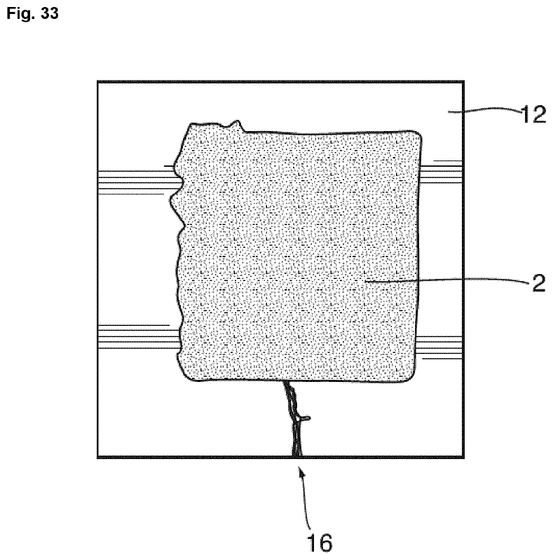

Once the textile conductive matrix was formed, the carbon-based dry electrode was fixed on the substrate through a thermal compression process as described in Example 8. A photograph of the fixed dry electrode on the knitted substrate is presented in FIG. 33.

### Tailoring

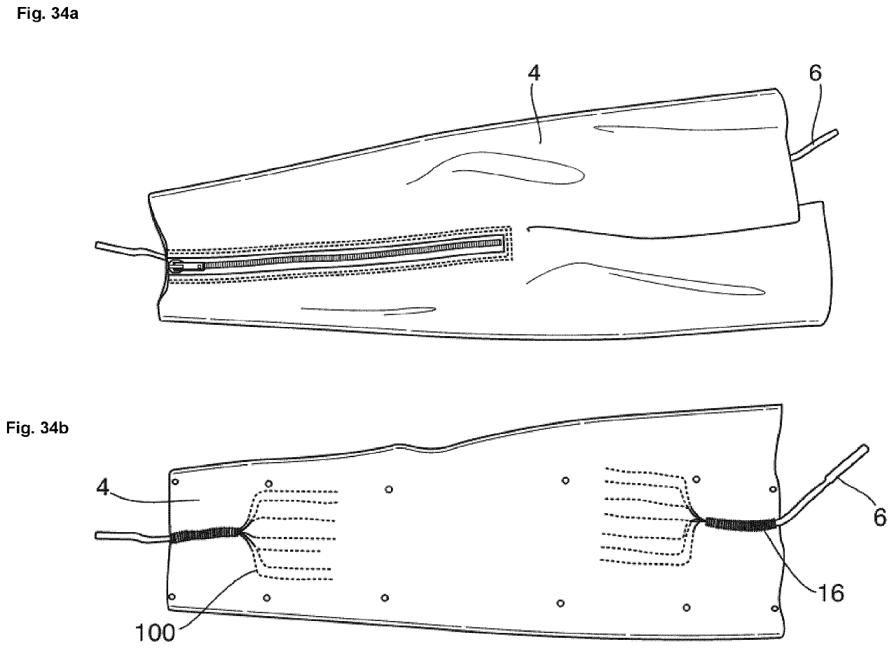

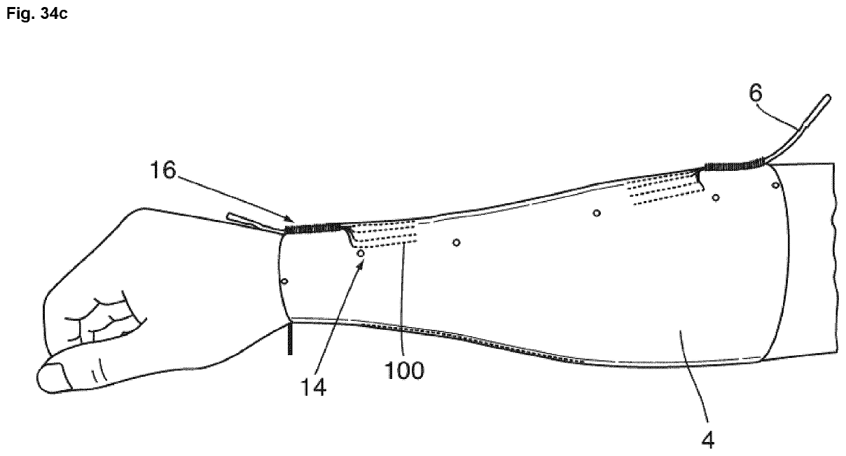

The last step of the manufacturing process consisted of tailoring the pattern to shape it into a sleeve and integrate the connectors. The leftover protruding textile conductive yarns from the conductive matrix were braided and inserted into a circular crimp connector, which was then threaded into shrink tubing. The resulting cables were fixed on the substrate using cording stitches. A zipper was sewn on two-thirds of the pattern, starting from the wrist part, to facilitate wearing. The remaining third was sewed with straight stitches. The prototype of the textile functional electrical stimulation sleeve prototype is presented in FIG. 34.

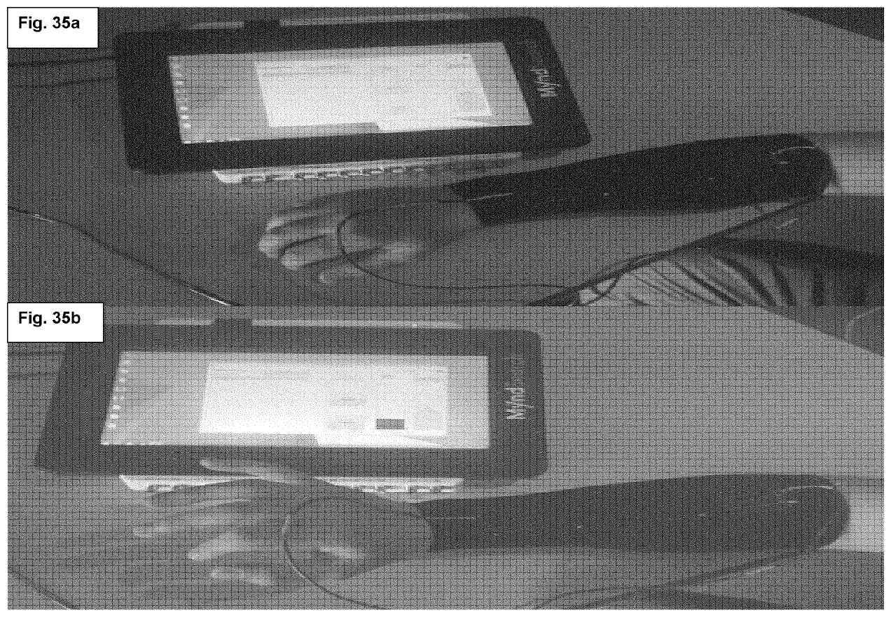

Experimental Protocol The aim was to provide a smart sleeve for functional electrical stimulation which provides a wrist extension through electrical stimulation without causing pain. The stimulation was delivered using MyndSearch® stimulator. FIG. 35 shows the experimental setup for both the wrist at rest and in extension. The study focused on the presence or absence of wrist flexion before pain occurs. It was conducted with a group of able bodied individuals.

### Results

Wrist flexion without pain was found for a subset of the participants, which was equivalent to the results achieved with the dry electrodes alone (i.e. without being integrated into the textile solution) as detailed in Example 4 above. The dry electrode manufacturing process and electrode integration into the garment did not alter the stimulation properties of the dry stimulation electrode. Thus, the forearm FES sleeve ensured a good skin/electrode interface and the textile conductive matrix enabled a homogeneous current distribution within the dry electrode as it was delivered to the skin.

It is to be understood that the application is not limited to the disclosed examples. To the contrary, the application is intended to cover various modifications and equivalent arrangements included within the spirit and scope of the appended claims.

All publications, patents and patent applications referred to are herein incorporated by reference in their entirety to the same extent as if each individual publication, patent or patent application was specifically and individually indicated to be incorporated by reference in its entirety.

The scope of the claims should not be limited by the preferred embodiments and examples but should be given the broadest interpretation consistent with the description as a whole.

### REFERENCES

[1]C. L. Lynch and M. R. Popovic, “Functional Electrical Stimulation,” IEEE Control Systems Magazine, vol. 28, no. 2, pp. 40-50, April 2008, doi: 10.1109/MCS.2007.914689.
[2]C. Marquez-Chin and M. R. Popovic, “Functional electrical stimulation therapy for restoration of motor function after spinal cord injury and stroke: a review,” BioMed Eng OnLine, vol. 19, no. 1, p. 34, December 2020, doi: 10.1186/s12938-020-00773-4.
[3]N. Kapadia, B. Moineau, and M. R. Popovic, “Functional Electrical Stimulation Therapy for Retraining Reaching and Grasping After Spinal Cord Injury and Stroke,” Front. Neurosci., vol. 14, 2020, doi: 10.3389/fnins.2020.00718.
[4]L. L. Baker, C. L. Wederich, D. R. McNeal, C. Newsam, and R. L. Waters, Neuro Muscular Electrical Stimulation: A Practical Guide, 4^{th} ed. Downey, CA: Los Amigos Research & Education Institute, Inc, 2000.
[5]T. Keller and A. Kuhn, “Electrodes for transcutaneous (surface) electrical stimulation,” J Autom Control, vol. 18, no. 2, pp. 35-45, 2008, doi: 10.2298/JAC0802035K.
[6] Brissot R, Gallien P, Le Bot M P, Beaubras A, Laisne D, Beillot J, Dassonville J. Clinical experience with functional electrical stimulation-assisted gait with parastep in spinal cord-injured patients, SPINE, 2000, 25, 4, 501-508, https://doi.org/10.1097/00007632-200002150-00018
[7] Thrasher T A, Flett H M, Popovic M R. Gait training regimen for incomplete spinal cord injury using functional electrical stimulation, Spinal Cord, 2006, 44, 357-361, https://doi.org/10.1038/sj.sc.3101864
[8]T. Keller and A. Kuhn, “Skin properties and the influence on electrode design for transcutaneous (surface) electrical stimulation,” in World Congress on Medical Physics and Biomedical Engineering, Sep. 7-12, 2009, Munich, Germany, vol. 25/9, O. Dössel and W. C. Schlegel, Eds. Berlin, Heidelberg: Springer Berlin Heidelberg, 2009, pp. 492-495. Doi: 10.1007/978-3-642-03889-1_131.
[9]A. Kuhn, T. Keller, M. Lawrence, and M. Morari, “The Influence of Electrode Size on Selectivity and Comfort in Transcutaneous Electrical Stimulation of the Forearm,” IEEE Trans. Neural Syst. Rehabil. Eng., vol. 18, no. 3, pp. 255-262, June 2010, doi: 10.1109/TNSRE.2009.2039807.
[10] Popovic M R, Keller T. Modular transcutaneous functional electrical stimulation system, Medical Engineering & Physics, 2007, 27, 81-92, https://doi.org/10.1016/j.medengphy.2004.08.016
[11] Popovic-Maneski L, Mateo S. MotiMove: Multi-purpose transcutaneous functional electrical stimulator, Artificial Organs, 2022, 46 (10), 1970-1979, https://doi.org/10.1111/aor.14379
[12] Kusche R, Kaufman S, Ryschka M. Dry electrodes for bioimpedance measurements-design, characterization and comparison, Biomed. Phys. Eng. Express, 2019, 5, 015001, https://doi.org/10.1088/2057-1976/aaea59
[13] Niu X, Gao X, Liu Y, Liu H. Surface bioelectric dry Electrodes: A review, Measurements, 2021, 183, 109774, (2021), https://doi.org/10.1016/Ij.measurement.2021.109774
[14] Lopes C, Veloso H, Hayes M, Cullinan M A, Vaz F. Nanostructured (Ti,Cu)N dry electrodes for advanced control of the neuromuscular activity, IEEE Sensors Journal, 2023, 23, 4, 3629, https://doi.org/10.1109/JSEN.2022.3232264
[15] Skrzetuska E, Michalak D, Krucinska I. Design and Analysis of Electrodes for Electrostimulation (TENS) Using the Technique of Film Printing and Embroidery, Sensors, 2021, 21, 4789, https://doi.org/10.3390/s21144789
[16] Euler L, Guo L, Persson N K. A review of textile-based electrodes developed for electrostimulation, Textile Research Journal, 2022, 92 (7-8), https://doi.org/0.1177/00405175211051949
[17] Keller T, Kuhn A. Electrodes for transcutaneous (surface) electrical stimulation, Journal of Automatic control, University of Belgrade, 2008, 18(2):35-45, https://doi.org/10.2298/JAC0802035K
[18]B. Moineau, C. Marquez-Chin, M. Alizadeh-Meghrazi, and M. R. Popovic, “Garments for functional electrical stimulation: Design and proofs of concept,” Journal of Rehabilitation and Assistive Technologies Engineering, vol. 6, p. 205566831985434, January 2019, doi: 10.1177/2055668319854340.
[19]B. Moineau, M. Myers, S. S. Ali, M. R. Popovic, and S. L. Hitzig, “End-user and clinician perspectives on the viability of wearable functional electrical stimulation garments after stroke and spinal cord injury,” Disability and Rehabilitation: Assistive Technology, pp. 1-10, October 2019, doi: 10.1080/17483107.2019.1668974.
[20]H. Zhou et al., “Stimulating the Comfort of Textile Electrodes in Wearable Neuromuscular Electrical Stimulation,” Sensors, vol. 15, no. 7, pp. 17241-17257, July 2015, doi: 10.3390/s150717241.
[21]L. Euler, L. Guo, and N.-K. Persson, “A review of textile-based electrodes developed for electrostimulation,” Textile Research Journal, vol. 92, no. 7-8, pp. 1300-1320, April 2022, doi: 10.1177/00405175211051949.
[22]M. Chu and H. E. Naguib, “Soft flexible conductive CNT nanocomposites for ECG monitoring,” Smart Materials and Structures, vol. 30, p. 065003, 2021.
[23]T. Chen et al., “Novel, Flexible, and Ultrathin Pressure Feedback Sensor for Miniaturized Intraventricular Neurosurgery Robotic Tools,” in IEEE Transactions on Industrial Electronics, vol. 68, no. 5, pp. 4415-4425, May 2021, doi: 10.1109/TIE.2020.2984427.
[24] Stewart A M, Pretty C G, Chen X. An Evaluation of the effect of stimulation and electrode type on bicep muscle for a voltage-controlled functional electrical stimulator, IFAC PapersOnLine, 2017, 50-1, 15109-15114, https://doi.org/10.1016/Ij.ifacol.2017.08.2242
[25] Yang K, Freeman C, Torah R, Beeby S, Tudor J. Screen printed fabric electrode array for wearable functional electrical stimulation, Sensors and Actuators A, 2014, Vol. 213, 108-115, https://doi.org/10.1016/j.sna.2014.03.025
[26] Poboroniuc M S, Irimia D C, Curteza A, Cretu V, Macovei L. Improved neuroprostheses by means of knitted textiles electrodes used for functional electrical stimulation, 2016 International Conference and Exposition on Electrical and Power Engineering, 2016, 20-22 Oct., 320-325, https://doi.org/10.1109/ICEPE.2016.7781355
[27] Kim H, Rho H, Lim D, Keong W. Characterization of embroidered textile-based electrode for EMG smart wear according to stitch technique, Fashion and Textiles, 2023, 10:32, https://doi.org/10.1186/s40691-023-00351-x
[28] Logothetis I, Fernandez-Garcia R, Troynikov O, Dabnichki P, Pirogova E, Gil I. Embroidered electrodes for bioelectrical impedance analysis: impact of surface area and stitch parameters, Meas. Sci. Technol., 2019, Vol. 30, 115103, https://doi.org/10.1088/1361-6501/ab3201
[29]S. Begum, H. Ullah, A. Kausar, M. Siddiq, and M. A. Aleem, “Fabrication of epoxy functionalized MWCNTs reinforced PVDF nanocomposites with high dielectric permittivity, low dielectric loss and high electrical conductivity,” Composites Science and Technology, vol. 167, pp. 497-506, October 2018, doi: 10.1016/j.compscitech.2018.08.041.
[30] Lin R, Kim H J, Achavananthadith S, Kurt S A, Tan S C C, Yao H, Tee B C K, Ho J S. Wireless battery-free body sensor networks using near-field-enabled colting, Nat. Commun, 2020, 11, 444, https://doi.org/10.1038/s41467-020-14311-2
[31] Ankhili A, Uz Zaman S, Tao X, Cochrane C, Koncar V, Coulon D. How to connect conductive flexible textile tracks to skin electrocardiography electrodes and protect them against washing, IEEE Sensors Journal, 2019, 19, 24, https://doi.org/10.1109/JSEN.2019.2938333
[32] Gamier B, Mariage P, Rault F, Cochrane C, Koncar V. Electronic-component less fully textile multiple resonant combiners for body-centric near field communication, Sci Rep, 2021, 11, 2159, https://doi.org/10.1038/s41598-021-81246-z
[33] Madeira Company®, Technical Datasheet, HC 12 silver-plated yarn, https://www.madeira.com/fileadmin/user upload/Downloads/Data_Sheets/Data_Sheet_HC_1 2__EN.pdf
[34] Uz Zaman S, Tao X, Cochrane C, Koncar V. Understanding washing damage to textile ECG dry skin electrodes, embroidered and fabric-based, set up of equivalent laboratory tests, Sensors, 2020, 20, 1272, https://doi.org/10.3390/s20051272

1. A reusable and transcutaneous dry electrode comprising:
i) a conductive material comprising a fluoropolymer matrix and conductive carbon nanoparticles dispersed in the matrix, and
ii) a conductor configured to contact and deliver an electrical pulse to the material from a stimulator,
wherein a dry tissue-contacting surface of the material is configured to deliver electrical stimulation directly to a tissue.

2. The electrode of claim 1, wherein the conductive material is non-metallic.

3. The electrode of claim 1 or 2, wherein the fluoropolymer matrix is polyvinylidene fluoride (PVDF) and/or the nanoparticles are carbon nanotubes (CNTs).

4. The electrode of any one of claims 1 to 3, wherein the nanoparticles are dispersed in the fluoropolymer matrix at about 5 to about 10 wt % of the matrix.

5. The electrode of any one of claims 1 to 4, wherein the nanoparticles have an aspect ratio of about 1 to about 600.

6. The electrode of claim 5, wherein the aspect ratio is about 130 to about 160.

7. The electrode of any one of claims 1 to 6, wherein the nanoparticles are interconnected.

8. The electrode of any one of claims 1 to 7, wherein impedance of the conductive material is about 1,000 to about 10,000Ω and/or sheet resistivity of the conductive material is at least 50 Ω·cm or about 50 to about 500 Ω·cm.

9. The electrode of claim 8, wherein the impedance is about 2,100 to about 2,800Ω and/or the sheet resistivity is about 100 to about 300 Ω·cm.

10. The electrode of any one of claims 1 to 9, wherein the conductive material has a width of about 50 to about 200 μm.

11. The electrode of claim 10, wherein the width is about 50 to about 100 μm.

12. The electrode of any one of claims 1 to 11, wherein the tissue is skin.

13. The electrode of claim 12, wherein the electrode is adapted for positioning on the skin in proximity to a selected muscle or group thereof.

14. The electrode of claim 13, wherein the electrical stimulation induces a contraction of the muscle or group thereof.

15. The electrode of any one of claims 1 to 14, wherein the electrical stimulation is functional electrical stimulation.

16. The electrode of any one of claims 1 to 15, wherein the electrode is disposed on an article or a garment.

17. The electrode of claim 16, wherein the electrode is disposed on an inside surface of the article or garment such to juxtapose the dry tissue-contacting surface of the electrode against the tissue.

18. The electrode of claim 16 or 17, wherein the electrode is affixed to the article or garment with an adhesive.

19. The electrode of any one of claims 1 to 18 for use with a high-rise voltage-regulated stimulator, wherein the electrode is configured to deliver an electrical pulse.

20. The electrode of claim 19 wherein the pulse is a biphasic electrical pulse that is symmetric or asymmetric.

21. The electrode of claim 19 or 20, wherein rise time of the pulse is about 10 to about 40 ns.

22. The electrode of any one of claims 19 to 21, wherein the pulse has a total width of about 8 to about 2,000 μs.

23. The electrode of claim 22, wherein the width is about 100 to about 1,000 μs.

24. The electrode of claim 23, wherein the width is about 300 to about 500 μs.

25. The electrode of any one of claims 20 to 24, wherein the pulse is asymmetric and comprises a first phase pulse having a first amplitude and a first width and a second phase pulse having a second amplitude and a second width.

26. The electrode of claim 25, wherein the first amplitude is about −0.04 to about −160 mA and/or the second amplitude is about 0.01 to about 40 mA.

27. The electrode of claim 26, wherein the first amplitude is about −0.4 to about −120 mA and/or the second amplitude is about 0.1 to about 30 mA.

28. The electrode of any one of claims 25 to 27, wherein the first width is about 200 to about 500 μs and/or the second width is about 800 to about 2,000 μs.

29. The electrode of any one of claims 25 to 28, wherein the first and second amplitudes are unequal.

30. The electrode of any one of claims 25 to 29, wherein the first and second widths are unequal.

31. A washable dry electrode article or garment comprising the electrode of any one of claims 1 to 30.

32. The article or garment of claim 31, wherein the electrode is disposed on a portion of an inside surface of the article or garment such to juxtapose the tissue-contacting surface of the conductive material against the tissue.

33. The article or garment of claim 32, wherein the electrode is affixed to the portion of the article or garment with an adhesive.

34. The article or garment of claim 31 or 32, wherein the conductor comprises an electrode-contacting segment configured for contacting the dry electrode, the electrode-contacting segment comprising at least one conductive yarn integrated into a knitted textile substrate.

35. The article or garment of claim 34, wherein the at least one conductive yarn is integrated into the textile substrate via sewing or embroidery.

36. The article or garment of claim 34 or 35 comprising at least two conductive yarns, wherein portions of the at least two conductive yarn are integrated into the textile substrate to form a pattern comprising a plurality of non-intersecting lines.

37. The article or garment of any one of claims 34-36, wherein the conductor further comprises a cable segment comprising non-integrated portions of the at least one or two conductive yarns configured for delivering electrical stimulation to the conductor electrode-contacting segment from a stimulator, optionally wherein the cable segment is secured to the textile substrate by an attachment means.

38. The article or garment of claim 36 or 37, wherein lengths of at least two of the plurality of non-intersecting lines are arranged substantially parallel relative to each other within the electrode-contacting segment.

39. The article or garment of any one of claims 34 to 38, wherein the textile substrate is or comprises polyester jersey fabric.

40. The article or garment of any one of claims 34 to 39, wherein the at least one or two conductive yarns are or comprise silver-plated yarns and/or have a linear resistance of at least 1 Ω/m or about 1 to about 1000 Ω/m.

41. The article or garment of any one of claims 34 to 40, wherein a protective layer is disposed on a non-skin-contacting surface of the article or garment to cover exposed portions of the at least one or two conductive yarns.

42. The article or garment of claim 41, wherein the protective layer is a fabric layer or a liquid silicone layer.

43. A dry electrode device for delivering electrical stimulation to a user comprising:
i) the article or garment as defined in any one of claims 31 to 42,
ii) a high-rise voltage-regulated stimulator, and
iii) a connector linking the conductor and the stimulator.

44. The device of claim 43, wherein the dry electrode is affixed to the article or garment with an adhesive.

45. The device of claim 43 or 44, wherein the connector is disposed on the article or garment.

46. The device of any one of claims 43 to 45, wherein the stimulator is portable.

47. The device of claim 46, wherein the stimulator is configured to be positioned within a portion of the article or garment.

48. Use of the dry electrode of any one of claims 1 to 30, the article or garment of any one of claims 31 to 42, or the device of any one of claims 43 to 47 for treating a mobility impairment in the user.

49. The use of claim 48 comprising:
i) positioning the article, garment, or at least two dry electrodes directly on the tissue of the user in proximity to a selected muscle or group thereof,
ii) connecting the conductor of the article, garment or the at least two dry electrodes to the stimulator, and
iii) delivering electrical stimulation from the stimulator to the conductor of the article or garment or to the at least two dry electrodes,
wherein the stimulation induces a contraction of the muscle or group thereof to treat the mobility impairment.

50. The use of claim 48 or 49, wherein the mobility impairment is paralysis.

51. A method of delivering electrical stimulation to a subject comprising:
i) positioning the article or garment as defined in any one of claims 31 to 42 or at least two dry electrodes as defined in any one of claims 1 to 30 directly on the tissue of the subject in proximity to a selected muscle or group thereof,
ii) connecting the conductors of the article, garment or the at least two dry electrodes to a high-rise voltage-regulated stimulator, and
iii) delivering an electrical pulse from the stimulator to the conductors of the article or garment or to the at least two dry electrodes,
wherein the stimulation induces a contraction of the muscle or group thereof.

52. The method of claim 51 wherein the pulse is a biphasic electrical pulse that is symmetric or asymmetric.

53. The method of claim 51 or 52, wherein rise time of the pulse is about 10 to about 40 ns.

54. The method of any one of claims 51 to 53, wherein the pulse has a total width of about 8 to about 2,000 μs.

55. The method of claim 54, wherein the width is about 100 to about 1,000 μs.

56. The method of claim 55, wherein the width is about 300 to about 500 μs.

57. The method of any one of claims 51 to 56, wherein the pulse is asymmetric and comprises a first phase pulse having a first amplitude and a first width and a second phase pulse having a second amplitude and a second width.

58. The method of claim 57, wherein the first amplitude is about −0.04 to about −160 mA and/or the second amplitude is about 0.01 to about 40 mA.

59. The method of claim 58, wherein the first amplitude is about −0.4 to about −120 mA and/or the second amplitude is about 0.1 to about 30 mA.

60. The method of any one of claims 57 to 59, wherein the first width is about 200 to about 500 μs and/or the second width is about 800 to about 2,000 μs.

61. The method of any one of claims 57 to 60, wherein the first and second amplitudes are unequal.

62. The method of any one of claims 57 to 61, wherein the first and second widths are unequal.

63. The method of any one of claims 51 to 62, wherein the pulse is delivered at a frequency of about 1 to about 100 Hz.

64. The method of claim 63, wherein the frequency is about 20 to about 40 Hz.

65. Use of the method of any one of claims 51 to 64 for treating a mobility impairment in the subject.

66. A method of manufacturing a washable dry electrode article or garment comprising:
A) fabricating the dry electrode of any one of claims 1 to 30, the steps comprising:
i) dispersing conductive carbon nanoparticles into a polymer solvent to yield a dispersion,
ii) dissolving fluoropolymer matrix in the dispersion to yield a blend,
iii) casting the blend,
iv) drying the casted blend,
v) compressing the blend,
vi) cooling the blend to yield a conductive material,
vii) cleaning the material, and
viii) disposing a conductor on or in the conductive material, wherein the conductor is configured to contact and deliver an electrical pulse to the material from a stimulator, and
B) disposing the dry electrode of (A) on or in an article or a garment, wherein the electrode is disposed on an inside surface of the article or garment such to juxtapose the tissue-contacting surface of the electrode against the tissue.

67. The method of claim 66, wherein the casting and/or compressing comprises transferring the blend into a mold.

68. The method of claim 66 or 67, wherein the solvent is dimethylformamide (DMF).

69. The method of any one of claims 66 to 68, wherein the dispersing is effected by sonication.

70. The method of any one of claims 66 to 69, wherein the dissolving, casting and/or drying further comprises heating the blend.

71. The method of claim 70, wherein the heating is effected within about 80 to about 200° C.

72. The method of any one of claims 66 to 71, wherein the cleaning is effected with water and/or ethanol.

73. The method of claim 66, wherein the disposing a conductor on the material according to (viii) comprises integrating at least one conductive yarn into a knitted textile substrate.

74. The method of claim 73, wherein the integrating comprises sewing or embroidering.

75. The method of claim 73 or 74, wherein the disposing the dry electrode according to (B) further comprises:
i) positioning a thermoadhesive on the textile substrate before the integrating of the at least one conductive yarn,
ii) positioning the dry electrode on the thermoadhesive, and
iii) affixing the thermoadhesive to the dry electrode and textile substrate using thermal compression,
wherein the thermal compression integrates a portion of the at least one conductive yarn into the dry electrode conductive material.

76. The method of claim 75, wherein the thermal compression comprises heating the dry electrode to a temperature of at least 180° C., optionally to a temperature between the range of 180° C. to 230° C.

77. The method of claim 76, wherein the thermal compression comprises applying a pressure between the range of 1 to 10 tons to the textile substrate, optionally applying about 1 ton of pressure followed by applying about 5 tons of pressure to the textile substrate.

78. The method of claim 77, wherein the pressure is applied for a time within the range of 5 to 10 minutes, optionally wherein the about 1 ton of pressure applied for about 5 minutes and the about 5 tons of pressure is applied for about 5 to about 7 minutes.

79. The method of any one of claims 73 to 78, wherein the textile substrate is or comprises polyester jersey fabric.

80. The method of any one of claims 73 to 79, wherein the at least one conductive yarn is or comprises silver-plated yarn and/or has a resistance of at least 1 Ω·m or about 1 to about 1000 Ω·m.

81. The method of any one of claims 73 to 80, wherein a protective layer is disposed on a non-skin-contacting surface of the article or garment to cover exposed portions of the at least one conductive yarn.

82. The method of claim 81, wherein the protective layer is a fabric layer or a liquid silicone layer.

83. Use of the dry electrode article or garment as manufactured by the method of any one of claims 66 to 82 for treating a mobility impairment in the user.

84. The use of claim 83, wherein the mobility impairment is paralysis.
